(19) United States
(12) Patent Application Publication
(10) Pub. No.: US 2025/0277013 A1
(43) Pub. Date: Sep. 4, 2025

(54) T CELL RECEPTOR DERIVED BINDING POLYPEPTIDES

(71) Applicants: Deutsches Krebsforschungszentrum Stiftung des öffentlichen Rechts, Heidelberg, (DE); Universität Heidelberg, Heidelberg, (DE)

(72) Inventors: Lukas BUNSE, Heidelberg (DE); Yu-Chan CHIH, Heidelberg (DE); Edward GREEN, Heidelberg (DE); Michael KILIAN, Heidelberg (DE); Christopher KRÄMER, Heidelberg (DE); Michael PLATTEN, Heidelberg (DE); Wolfgang WICK, Heidelberg (DE)

(73) Assignees: Deutsches Krebsforschungszentrum Stiftung des öffentlichen Rechts, Heidelberg (DE); Universität Heidelberg, Heidelberg (DE)

(21) Appl. No.: 18/862,491

(22) PCT Filed: May 3, 2023
(86) PCT No.: PCT/EP2023/061716

§ 371 Date: Nov. 1, 2024

(30) Foreign Application Priority Data
May 4, 2022 (EP) 22171669.9

(51) Int. Cl. C07K 14/725; A61K 38/00; A61P 35/00; G01N 33/574

(52) U.S. Cl. CPC C07K 14/7051; A61P 35/00; G01N 33/57407; A61K 38/00; G01N 2333/7051

(57) ABSTRACT

The present invention relates to a binding polypeptide comprising a first variable T cell receptor (TCR) domain and a second variable TCR domain wherein (i) the complementarity determining region 3 (CDR3) of the first variable TCR domain comprises, preferably consists of, the amino acid sequence of SEQ ID NO: 1 or a sequence at least 80% identical thereto; and/or wherein the CDR3 of the second variable TCR domain comprises, preferably consists of, the amino acid sequence of SEQ ID NO:2 or a sequence at least 80% identical thereto; or wherein (ii) the CDR3 of the first variable TCR domain comprises, preferably consists of, the amino acid sequence of SEQ ID NO:3 or a sequence at least 80% identical thereto; and/or wherein the CDR3 of the second variable TCR domain comprises, preferably consists of, the amino acid sequence of SEQ ID NO:4 or a sequence at least 80% identical thereto; and to polynucleotides, host cells, methods, uses, kits, and devices related thereto.

The present invention relates to a binding polypeptide comprising a first variable T cell receptor (TCR) domain and a second variable TCR domain wherein (i) the complementarity determining region 3 (CDR3) of the first variable TCR domain comprises, preferably consists of, the amino acid sequence of SEQ ID NO: 1 or a sequence at least 80% identical thereto; and/or wherein the CDR3 of the second variable TCR domain comprises, preferably consists of, the amino acid sequence of SEQ ID NO:2 or a sequence at least 80% identical thereto; or wherein (ii) the CDR3 of the first variable TCR domain comprises, preferably consists of, the amino acid sequence of SEQ ID NO:3 or a sequence at least 80% identical thereto; and/or wherein the CDR3 of the second variable TCR domain comprises, preferably consists of, the amino acid sequence of SEQ ID NO:4 or a sequence at least 80% identical thereto; and to polynucleotides, host cells, methods, uses, kits, and devices related thereto.

Adoptive T cell therapy using genetically engineered T cells expressing a chimeric antigen receptor (CAR) against the B cell antigen CD19 has achieved remarkable regressions in leukemia and lymphoma patients. Translation of this cellular concept to solid tumors has proven to be difficult, a major hurdle being the identification of suitable cell surface antigens. On the other hand, intracellular tumor-associated antigens (TAAs) can also be processed and presented on major histocompatibility complexes to T cells and can therefore be exploited as T cell targets. Despite successful induction or amplification of T cell responses against TAAs by therapeutic vaccinations, the level of cellular effector immune responses required to control tumor growth has not been achieved (e.g. Hilf et al., Nature 565:240-245 (2019); Keskin et al., Nature 565:234-239 (2019)).

Tumor-infiltrating leukocyte (TIL) therapy is an alternative therapeutic option to amplify spontaneous T cell responses ex vivo. However, prior to re-infusion to patients, unspecific cytokine-driven in vitro expansion of TILs inevitably leads to a loss of potentially TAA-reactive T cell clones, diminishing the efficacy of cellular therapies. Here, T cell receptor (TCR)-transgenic cellular therapy provides a valuable alternative since transgenic cells target known and defined antigens, TAA-reactive transgenic cells can be additionally genetically modified to enhance T cell responses, and in contrast to TIL therapy, the re-infusion of a monoclonal TCR-transgenic T cell product allows exact enumeration of truly tumor-reactive T cells.

Gliomas, which generally are tumors with low mutational burden, contain only 30-50 non-synonymous mutations. Recently, two multinational and multicenter glioblastoma (WHO grade IV gliomas) vaccine consortia, The Glioma Actively Personalized Vaccine Consortium (GAPVAC) and The NeoVax Consortium, respectively, independently reported successful induction of highly personalized TAA-specific but also tumor-specific antigen (TSA)-specific T cell responses in glioblastoma patients (Hilf et al., loc. cit; Keskin et al., loc. cit.). Both phase 1 trials were able to demonstrate homing of vaccine-induced T cells to brain tumors. Similarly, a multicenter, single-arm, open-label, first-in-humans phase I trial that was carried out in 33 patients with newly diagnosed World Health Organization grade 3 and 4 mutant isocitrate dehydrogenase 1 (IDH1R132H)-positive astrocytomas (Neurooncology Working Group of the German Cancer Society trial 16 (NOA16), NCT02454634) provided proof-of-concept that target-specific T helper cells are in principle able to home into gliomas (Platten et al., Nature 592:463-468 (2021)). At the same time, evidence from these studies suggest that the magnitude of vaccine-induced specific T cell responses can still be improved.

Thus, there is still a need for improved cancer treatments. The technical problem underlying the present invention can be seen as the provision of means and methods for complying with the aforementioned needs. The technical problem is solved by the embodiments characterized in the claims and herein below.

In accordance, the present invention relates to a binding polypeptide comprising a first variable T cell receptor (TCR) domain and a second variable TCR domain.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”. In accordance, expressions relating to one item of a plurality, unless otherwise indicated, preferably relate to at least one such item, more preferably a plurality thereof; thus, e.g. identifying “a cell” relates to identifying at least one cell, preferably to identifying a multitude of cells.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The methods specified herein below, preferably, are in vitro methods. The method steps may, in principle, be performed in any arbitrary sequence deemed suitable by the skilled person, but preferably are performed in the indicated sequence; also, one or more, preferably all, of said steps may be assisted or performed by automated equipment. Moreover, the methods may comprise steps in addition to those explicitly mentioned above.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The degree of identity (e.g. expressed as “% identity”) between two biological sequences, preferably DNA, RNA, or amino acid sequences, can be determined by algorithms well known in the art. Preferably, the degree of identity is determined by comparing two optimally aligned sequences over a comparison window, where the fragment of sequence in the comparison window may comprise additions or deletions (e.g., gaps or overhangs) as compared to the sequence it is compared to for optimal alignment. The percentage is calculated by determining, preferably over the whole length of the polynucleotide or polypeptide, the number of positions at which the identical residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr., Madison, WI), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. In the context of biological sequences referred to herein, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “fragment” of a biological macromolecule, preferably of a polynucleotide or polypeptide, is used herein in a wide sense relating to any sub-part, preferably subdomain, of the respective biological macromolecule comprising the indicated sequence, structure and/or function. Thus, the term includes sub-parts generated by actual fragmentation of a biological macromolecule, but also sub-parts derived from the respective biological macromolecule in an abstract manner, e.g. in silico. Thus, as used herein, an Fc or Fab fragment, but also e.g. a single-chain antibody, a bispecific antibody, and a nanobody may be referred to as fragments of an immunoglobulin.

Unless specifically indicated otherwise herein, the compounds specified, in particular the polynucleotides and polypeptides, may be comprised in larger structures, e.g. may be covalently or non-covalently linked to further sequences, carrier molecules, retardants, and other excipients. In particular, polypeptides as specified may be comprised in fusion polypeptides comprising further peptides, which may serve e.g. as a tag for purification and/or detection, as a linker, or to extend the in vivo half-life of a compound. The term “detectable tag” refers to a stretch of amino acids, which are added to or introduced into the fusion polypeptide; preferably, the tag is added C-or N-terminally to the fusion polypeptide. Said stretch of amino acids preferably allows for detection of the polypeptide by a specific binding partner, e.g. an antibody, which specifically recognizes the tag; or it preferably allows for forming a functional conformation, such as a chelator; or it preferably allows for visualization, e.g. in the case of fluorescent tags. Preferred detectable tags are the Myc-tag, FLAG-tag, 6-His-tag, HA-tag, GST-tag or a fluorescent protein tag, e.g. a GFP-tag. These tags are all well known in the art. Other further peptides preferably comprised in a fusion polypeptide comprise further amino acids or other modifications, which may serve as mediators of secretion, as mediators of blood-brain-30 barrier passage, as cell-penetrating peptides, and/or as immune stimulants. Further polypeptides or peptides to which the polypeptides may be fused are signal and/or transport sequences, e.g. an IL-2 signal sequence, and linker sequences.

The term “polypeptide”, as used herein, refers to a molecule comprising several, typically at least 20, amino acids that are covalently linked to each other by peptide bonds. Molecules consisting of less than 20 amino acids covalently linked by peptide bonds are usually considered to be “peptides”. Preferably, the polypeptide comprises of from 50 to 1000, more preferably of from 60 to 1000, still more preferably of from 70 to 500, most preferably of from 80 to 400 amino acids. The polypeptide may be a complex of more than one amino acid chains, i.e. may be a multimer, e.g. a dimer, a trimer, and the like; in such case, the complex of more than one amino acid chains may also be referred to as an “polypeptide oligomer” or as a “protein complex”. Preferably, the complex of more than one amino acid chains is a hetero-multimer, more preferably a hetero-dimer, preferably comprising at least one first variable TCR domain and at least one second variable TCR domain in a non-covalent complex. Also, the polypeptide may comprise additional, non-peptidic structures, such as at least one glycosylation, lipid conjugation, and the like. More preferably, the polypeptide as specified comprises all structural components as indicated comprised in one continuous covalent peptide chain, thus, the polypeptide preferably is or is comprised in a fusion polypeptide. Unless specifically indicated otherwise, reference to specific polypeptides herein preferably includes polypeptide variants.

As used herein, the term “polypeptide variant” relates to any chemical molecule comprising at least one polypeptide as specified herein, having the indicated activity, but differing in structure from said specific polypeptide. Preferably, the polypeptide variant comprises a polypeptide having a contiguous amino acid sequence corresponding to at least 50%, preferably at least 75%, more preferably at least 85%, even more preferably at least 90%, most preferably at least 95%, of the amino acid sequence of the polypeptide specifically indicated. Moreover, it is to be understood that a polypeptide variant as referred to in accordance with the present invention shall have an amino acid sequence which differs due to at least one amino acid substitution, deletion and/or addition, wherein the amino acid sequence of the variant is still, preferably, at least 70%, more preferably at least 80%, even more preferably at least 90%, even more preferably at least 95%, still more preferably at least 98%, most preferably at least 99%, identical with the amino acid sequence of the specific polypeptide. The degree of identity between two amino acid sequences can be determined by algorithms well known in the art and as described herein above. Polypeptide variants referred to above may be allelic variants or any other species-specific homologs, paralogs, or orthologs. Moreover, the polypeptide variants referred to herein include fragments of the specific polypeptides or the aforementioned types of polypeptide variants as long as these fragments and/or variants have the biological activity as specified. Such fragments may be or may be derived from, e.g., degradation products or splice variants of the polypeptides. Further included are variants, which differ due to posttranslational modifications such as phosphorylation, glycosylation, ubiquitinylation, sumoylation, or myristylation, by including non-natural amino acids, and/or by being peptidomimetics.

The term “polynucleotide”, as used herein, refers to a linear or circular nucleic acid molecule. The polynucleotide of the present invention shall be provided, preferably, either as an isolated polynucleotide (i.e. isolated from its natural context) or in genetically modified form, preferably comprising at least one heterologous sequence. The term encompasses single-as well as double-stranded polynucleotides. Moreover, comprised are also chemically modified polynucleotides including naturally occurring modified polynucleotides such as glycosylated or methylated polynucleotides or artificially modified derivatives such as biotinylated polynucleotides, locked nucleic acids, and the like. The polynucleotides of the invention have the activity of encoding a binding polypeptide as specified herein. Methods for testing whether a given polynucleotide has the aforesaid activity are known in the art and are described herein below. Unless specifically indicated otherwise, reference to specific polynucleotides herein preferably includes polynucleotide variants.

The term “polynucleotide variant”, as used herein, relates to a variant of a polynucleotide referred to herein comprising a nucleic acid sequence characterized in that the sequence can be derived from the aforementioned specific nucleic acid sequence by at least one nucleotide substitution, addition and/or deletion, wherein the polynucleotide variant shall have the activity as specified for the specific polynucleotide. Preferably, said polynucleotide variant is an ortholog, a paralog, or another homolog of the specific polynucleotide. Also preferably, said polynucleotide variant is or is derived from a non-naturally occurring allele of the specific polynucleotide. Polynucleotide variants also encompass polynucleotides comprising a nucleic acid sequence, which is capable of hybridizing to the aforementioned specific polynucleotides, preferably, under stringent hybridization conditions. These stringent conditions are known to the skilled worker and can be found in Current Protocols in Molecular Biology, John Wiley & Sons, N.Y. (1989), 6.3.1-6.3.6. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Alternatively, polynucleotide variants are obtainable by PCR-based techniques such as mixed oligonucleotide primer-based amplification of DNA. Further, variants include polynucleotides comprising nucleic acid sequences, which are at least 70%, preferably at least 80%, more preferably at least 90%, even more preferably at least 95%, still more preferably at least 98%, most preferably at least 99%, identical to the specifically indicated nucleic acid sequences. Moreover, also encompassed are polynucleotides, which comprise nucleic acid sequences encoding amino acid sequences, which are at least 70%, more preferably at least 80%, even more preferably at least 90%, even more preferably at least 95%, still more preferably at least 98, most preferably at least 99%, identical to the amino acid sequences specifically indicated. The percent identity values are, preferably, calculated over the entire amino acid or nucleic acid sequence region, preferably as specified herein above. The polynucleotides of the present invention either consist, essentially consist of, or comprise the aforementioned nucleic acid sequences. Thus, they may contain further nucleic acid sequences as well. Specifically, the polynucleotides of the present invention may encode fusion proteins wherein one partner of the fusion protein is a polypeptide being encoded by a nucleic acid sequence recited above. Also, the polynucleotide may be comprised in a vector.

The term “vector”, preferably, encompasses phage, plasmid, viral or retroviral vectors as well artificial chromosomes, such as bacterial or yeast artificial chromosomes. Moreover, the term also relates to targeting constructs, which allow for random or site-directed integration of the targeting construct into genomic DNA. Such target constructs, preferably, comprise DNA of sufficient length for either homologous or heterologous recombination as described in detail below. The vector encompassing the polynucleotide of the present invention, preferably, further comprises selectable markers for propagation and/or selection in a host. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerenes. Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host/cells. More preferably, in the vector of the invention the polynucleotide is operatively linked to expression control sequences allowing expression in prokaryotic or eukaryotic cells or isolated fractions thereof, i.e. preferably, the polynucleotide is comprised in an expression vector. Expression of said polynucleotide comprises transcription of the polynucleotide into an RNA. Regulatory elements ensuring expression in eukaryotic cells, preferably mammalian cells, are well known in the art. They, preferably, comprise regulatory sequences ensuring initiation of transcription and, optionally, poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Moreover, inducible expression control sequences may be used in an expression vector encompassed by the present invention. Such inducible vectors may comprise tet or lac operator sequences or sequences inducible by heat shock or other environmental factors. Suitable expression control sequences are well known in the art. Beside elements, which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pBluescript (Stratagene), pCDM8, pRc/CMV, pcDNA1, pcDNA3 (InVitrogene) or pSPORT1 (GIBCO BRL). Preferably, said vector is an expression vector and a gene transfer or targeting vector. Expression vectors derived from viruses such as retroviruses, vaccinia virus, adeno-associated virus, herpes viruses, or bovine papilloma virus, may be used for delivery of polynucleotides or vectors into targeted cell population. Methods, which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

The term “specific binding” is understood by the skilled person. Preferably, specific binding relates to a binding in which the affinity of the binding polypeptide to a peptide, preferably a peptide as specified elsewhere herein, is at least tenfold, preferably at least 100fold, most preferably at least 1000fold, higher than for any non-cognate (poly) peptide. Accordingly, the dissociation constant (K_{d}) of any binding polypeptide/non-cognate peptide complex preferably is at least 10⁻⁶ mol/L, more preferably at least 10⁻⁵ mol/l, most preferably at least 10⁻⁴ mol/L.

The term “binding polypeptide”, as used herein, relates to a polypeptide having the structural elements as specified herein and having the activity of binding its cognate peptide, preferably specifically binding said cognate peptide, more preferably specifically binding said cognate peptide when presented on an MHC class I molecule, preferably an HLA-A molecule, more preferably an HLA-A*02 molecule. Preferably, said cognate peptide comprises, more preferably consists of, the amino acid sequence MIWEHNVEV (SEQ ID NO:14) or comprises, more preferably consists of, the amino acid sequence NLDTLMTYV (SEQ ID NO:13).

The term “T cell receptor”, abbreviated as “TCR”, as used herein, relates to a polypeptide complex on the surface of T cells mediating recognition of antigenic peptides presented by target cells, preferably in the context of MHC molecules or MHC-related molecules such as MR1 or CD1, more preferably in the context of MHC molecules, still more preferably in the context of MHC class I or MHC class II molecules, most preferably in the context of MHC class I molecules. Typically, the TCR comprises one TCR-alpha chain and one TCR-beta chain, i.e. is an alpha/beta chain heterodimer. The TCR may, however, also comprise a TCR gamma and a TCR delta chain instead of the TCR alpha and beta chains. In accordance with usual nomenclature, the complex consisting of an alpha and a beta chain or a gamma and a delta chain is referred to as “T cell receptor” or “TCR” herein, the alpha and/or beta chain and the gamma and/or delta chains commonly or singly being referred to a “TCR polypeptide” or “TCR polypeptides”, whereas the polypeptide complex comprising a TCR and accessory polypeptides, such as CD3 and CD247, is referred to as “T cell receptor complex”, abbreviated as “TCR complex”. Each TCR polypeptide comprises several polypeptide domains, i.e. a transmembrane region (transmembrane domain), a constant region (constant domain), a joining region (constant domain), and a variable region (variable domain), the variable region of each TCR alpha, beta, gamma, or delta chain comprising three complementarity determining regions (CDRs), referred to as CDR1, CDR2, and CDR3, respectively. As referred to herein, a variable region of a TCR polypeptide is referred to as “variable T cell receptor domain”, which may be abbreviated as “variable TCR domain”. Nomenclatures for numbering amino acids in TCR domains or in immunoglobulin-superfamily polypeptides in general are known to the skilled person, e.g., the nomenclature of the international ImMunoGeneTics information system® (IMGT numbering scheme, Lefranc et al., Dev Comparative Immunol 27:55 (2003)), the Kabat numbering, and the like. Preferably, the numbering scheme is the IMGT numbering scheme and is used for amino acid numbering herein. As is known to the skilled person, in TCRs, binding specificity is essentially determined by the variable domain and specifically be the CDR3 region of the variable region. Thus, a TCR having the activity of binding a specific cognate peptide may be structurally defined by the amino acid sequences of the CDR3s of its variable domains.

The binding polypeptide comprises a first variable TCR domain and said second variable TCR domain. Preferably, the first variable TCR domain and the second variable TCR domain are comprised in a fusion polypeptide, i.e., preferably, in a continuous peptide chain. Also preferably, the first variable TCR domain is covalently connected to a first constant TCR domain, and/or the second variable TCR domain is covalently connected to a second constant TCR domain. Preferably, the first variable TCR domain and the second variable TCR domain are covalently connected to a transmembrane domain, preferably a TCR transmembrane domain. Thus, the binding polypeptide may be a TCR, preferably comprising al structural elements of a TCR as indicated elsewhere herein. Also preferably, the first variable TCR domain and the second variable TCR domain are covalently connected to a signaling domain. Thus, the binding polypeptide may be a chimeric antigen receptor (CAR). CARs and construction principles thereof are known in the art. Also preferably, the first variable TCR domain and said second variable TCR domain are covalently connected to an Fc domain of an immunoglobulin; thus, the binding polypeptide may preferably be an immunomodulatory compound. Also preferably, the binding polypeptide is a soluble TCR, preferably is a soluble TCR tetramer. Furthermore, the binding polypeptide may comprise a label, such as a colored and/or fluorescent dye; and/or an effector compound, such as a chemotherapeutic agent, a radioactive compound, and the like.

Preferably, the variable TCR domains of the binding polypeptide comprise CDR3 sequences mediating or contributing to the activity as specified herein above. Thus, preferably, (i) the complementarity determining region 3 (CDR3) of the first variable TCR domain comprises, preferably consists of, the amino acid sequence of SEQ ID NO:1 or a sequence at least 80% identical thereto; and/or the CDR3 of the second variable TCR domain comprises, preferably consists of, the amino acid sequence of SEQ ID NO:2 or a sequence at least 80% identical thereto; or (ii) the CDR3 of the first variable TCR domain comprises, preferably consists of, the amino acid sequence of SEQ ID NO:3 or a sequence at least 80% identical thereto; and/or the CDR3 of the second variable TCR domain comprises, preferably consists of, the amino acid sequence of SEQ ID NO:4 or a sequence at least 80% identical thereto. More preferably, (i) the CDR3 of the first variable TCR domain comprises, preferably consists of, the amino acid sequence of SEQ ID NO:1 or a sequence at least 80% identical thereto; and the CDR3 of the second variable TCR domain comprises, preferably consists of, the amino acid sequence of SEQ ID NO:2 or a sequence at least 80% identical thereto; or (ii) the CDR3 of the first variable TCR domain comprises, preferably consists of, the amino acid sequence of SEQ ID NO:3 or a sequence at least 80% identical thereto; and the CDR3 of the second variable TCR domain comprises, preferably consists of, the amino acid sequence of SEQ ID NO:4 or a sequence at least 80% identical thereto. Even more preferably, (i) the complementarity determining region 3 (CDR3) of the first variable TCR domain comprises, preferably consists of, the amino acid sequence of SEQ ID NO:1; and/or the CDR3 of the second variable TCR domain comprises, preferably consists of, the amino acid sequence of SEQ ID NO:2; or (ii) the CDR3 of the first variable TCR domain comprises, preferably consists of, the amino acid sequence of SEQ ID NO: 3; and/or the CDR3 of the second variable TCR domain comprises, preferably consists of, the amino acid sequence of SEQ ID NO:4.

More preferably, the first variable TCR domain comprises, preferably consists of, the amino acid sequence of SEQ ID NO:5 or an amino acid sequence at least 70%, identical to SEQ ID NO: 5 and/or the second variable TCR domain comprises, preferably consists of, the amino acid sequence of SEQ ID NO:6 or an amino acid sequence at least 70% identical to SEQ ID NO:6; or the first variable TCR domain comprises, preferably consists of, the amino acid sequence of SEQ ID NO:7 or an amino acid sequence at least 70% identical to SEQ ID NO:7 and/or the second variable TCR domain comprises, preferably consists of, the amino acid sequence of SEQ ID NO:8 or an amino acid sequence at least 70% identical to SEQ ID NO:8. Even more preferably, the first variable TCR domain comprises, preferably consists of, the amino acid sequence of SEQ ID NO:5 and/or the second variable TCR domain comprises, preferably consists of, the amino acid sequence of SEQ ID NO:6, or the first variable TCR domain comprises, preferably consists of, the amino acid sequence of SEQ ID NO:7 and/or wherein said second variable TCR domain comprises, preferably consists of, the amino acid sequence of SEQ ID NO:8.

Preferably, the amino acid sequence of SEQ ID NO:5 is encoded by the nucleic acid sequence of SEQ ID NO:9, and the amino acid sequence of SEQ ID NO:6 is encoded by the nucleic acid sequence of SEQ ID NO:10. Also preferably, the amino acid sequence of SEQ ID NO:7 is encoded by the nucleic acid sequence of SEQ ID NO: 11 and the amino acid sequence of SEQ ID NO: 8 is encoded by the nucleic acid sequence of SEQ ID NO:12. As the skilled person is aware of and as specified herein above, a given amino acid sequence may be encoded by a multitude of nucleic acid sequences due to the degeneracy of the genetic code; thus, a skilled person is able to provide appropriate coding sequences, which may be e.g. codon optimized for a host cell from a particular species.

Advantageously, it was found in the work underlying the present invention that the binding polypeptides as specified are particularly well suited for specific binding to cancer cells, in particular cancer cells expressing PTPRZ1 and/or NLGN4X and therefore presenting peptides with amino acid sequences as shown in SEQ ID NO:13 and/or NO: 14. Thus, the binding polypeptides provided herein are particularly suitable for cancer therapy, in particular T cell therapy of glioma.

The definitions made above apply mutatis mutandis to the following. Additional definitions and explanations made further below also apply for all embodiments described in this specification mutatis mutandis.

The present invention also relates to a polynucleotide encoding at least one of a first variable TCR domain and a second variable TCR domain of a binding polypeptide of the present invention, and to a vector comprising said polynucleotide.

The term “polynucleotide” which preferably includes polynucleotide variants, has been specified herein above, as have preferred nucleic acid sequences. Preferably, the polynucleotide encodes a first variable TCR domain and a second variable TCR domain of a binding polypeptide, more preferably encodes a fusion polypeptide comprising said first variable TCR domain and said second variable TCR domain. Preferably, the polynucleotide is an expression construct, i.e. preferably causing expression of at least one of a first variable TCR domain and a second variable TCR domain of a binding polypeptide in a suitable host cell.

The term vector has been specified herein above as well. Preferably, the vector is an expression vector.

The present invention also relates to a host cell comprising the binding polypeptide of the present invention, the polynucleotide of the present invention, and/or the vector of the present invention.

As used herein, the term “host cell” relates to any cell capable of receiving and, preferably, expressing a binding polypeptide, polynucleotide, or vector as specified. Preferably, the host cell is a bacterial cell, more preferably a cell of a common laboratory bacterial strain known in the art, most preferably an Escherichia strain, in particular an E. coli strain. Also preferably, the host cell is a eukaryotic cell, preferably a yeast cell, e.g. a cell of a strain of baker's yeast, or is an animal cell. More preferably, the host cell is an insect cell or a mammalian cell, in particular a mouse or rat cell. Most preferably, the host cell is a human cell. Preferably, the host cell is a T cell, more preferably a CD8+ T cell or a CD4+ T cell, more preferably a CD8+ T cell. As the skilled person understands, a CD8 TCR is preferably expressed in a CD8+ T cell, and a CD4 TCR is preferably expressed in a CD4+ T cell. Also preferably, the host cell is capable of presenting on its surface a binding polypeptide as specified herein, preferably encoded by a polynucleotide and/or vector as specified herein.

The present invention also relates to a binding polypeptide of the present invention, a polynucleotide of the present invention, a vector of the present invention, and/or a host cell of the present invention for use in medicine, in particular for use in treating and/or preventing cancer. In accordance, the binding polypeptide, the polynucleotide, the vector, and/or the host cell, may be formulated as pharmaceutical composition.

The present invention also relates to a method of treating and/or preventing cancer in a subject, said method comprising
(a) contacting said subject with a binding polypeptide of the present invention, a polynucleotide of the present invention, a vector of the present invention, and/or a host cell of the present invention, and
(b) thereby treating cancer in said subject.

The term “cancer”, as used herein, relates to a disease of an animal, including man, characterized by uncontrolled growth by a group of body cells (“cancer cells”). This uncontrolled growth may be accompanied by a formation of a cell mass of cancer cells (tumor), by intrusion into and destruction of surrounding tissue (infiltration) and possibly spread of cancer cells to other locations in the body (metastasis). Preferably, also included by the term cancer is a recurrence of a cancer (relapse). Thus, preferably, the cancer is a solid cancer, including a primary tumor, a metastasis, and a relapse thereof. The cancer may, however, also be a non-solid cancer. Preferably, the cancer is selected from the list consisting of acute lymphoblastic leukemia, acute myeloid leukemia, adrenocortical carcinoma, aids-related lymphoma, anal cancer, appendix cancer, astrocytoma, atypical teratoid, basal cell carcinoma, bile duct cancer, bladder cancer, brain stem glioma, breast cancer, burkitt lymphoma, carcinoid tumor, cerebellar astrocytoma, cervical cancer, chordoma, chronic lymphocytic leukemia, chronic myelogenous leukemia, colon cancer, colorectal cancer, craniopharyngioma, endometrial cancer, ependymoblastoma, ependymoma, esophageal cancer, extracranial germ cell tumor, extragonadal germ cell tumor, extrahepatic bile duct cancer, fibrosarcoma, gallbladder cancer, gastric cancer, gastrointestinal stromal tumor, gestational trophoblastic tumor, hairy cell leukemia, head and neck cancer, hepatocellular cancer, hodgkin lymphoma, hypopharyngeal cancer, hypothalamic and visual pathway glioma, intraocular melanoma, kaposi sarcoma, laryngeal cancer, medulloblastoma, medulloepithelioma, melanoma, merkel cell carcinoma, mesothelioma, mouth cancer, multiple endocrine neoplasia syndrome, multiple myeloma, mycosis fungoides, nasal cavity and paranasal sinus cancer, nasopharyngeal cancer, neuroblastoma, non-hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, papillomatosis, paranasal sinus and nasal cavity cancer, parathyroid cancer, penile cancer, pharyngeal cancer, pheochromocytoma, pituitary tumor, pleuropulmonary blastoma, primary central nervous system lymphoma, prostate cancer, rectal cancer, renal cell cancer, retinoblastoma, rhabdomyosarcoma, salivary gland cancer, sézary syndrome, small cell lung cancer, small intestine cancer, soft tissue sarcoma, squamous cell carcinoma, squamous neck cancer, testicular cancer, throat cancer, thymic carcinoma, thymoma, thyroid cancer, urethral cancer, uterine sarcoma, vaginal cancer, vulvar cancer, waldenström macroglobulinemia, and wilms tumor. More preferably, the cancer is a solid cancer, a metastasis, or a relapse thereof. More preferably, said cancer is a brain cancer, preferably a glioma, more preferably a glioblastoma.

The terms “treating” and “treatment” refer to an amelioration of a disease or disorder referred to herein or the symptoms accompanied therewith to a significant extent; as used herein, the term includes prevention of deterioration of a disease, disorder, or symptoms associated therewith. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well-known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 10%, at least 20% at least 50% at least 60%, at least 70%, at least 80%, or at least 90% of the subjects of a given cohort or population. Preferably, treating comprises activating an immune response against an agent causing or mediating disease, i.e. against cancer cells. Preferably, treating cancer is reducing tumor and/or cancer cell burden in a subject. As will be understood by the skilled person, effectiveness of treatment of e.g. cancer is dependent on a variety of factors including, e.g. cancer stage and cancer type. Also preferably, cancer treatment further comprises at least one of surgery, chemotherapy, and radiotherapy.

The terms “preventing” and “prevention” refer to retaining health with respect to the diseases or disorders referred to herein for a certain period of time in a subject. It will be understood that said period of time may be dependent on the amount of the drug compound which has been administered and individual factors of the subject discussed elsewhere in this specification. It is to be understood that prevention may not be effective in all subjects treated with the compound according to the present invention. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context, which normally, i.e. without preventive measures according to the present invention, would develop a disease or disorder as referred to herein. Whether a portion is statistically significant can be determined without further ado by the person skilled in the art using various well-known statistic evaluation tools discussed elsewhere in this specification. In the context of cancer treatment, preventing in particular relates to preventing cancer development, preventing metastasis formation, and/or preventing relapse, preferably relates to preventing metastasis formation and/or preventing relapse.

Preferably, treating and/or preventing cancer comprises administration of host cells, preferably of T cells as specified herein above, producing a binding polypeptide as specified herein, to a subject to be treated. Methods of conferring to a host cell the capability of producing a binding polypeptide are, in principle, known in the art, and include in particular introduction of at least one expression construct, e.g. an expression vector, into said host cell. Appropriate methods depend on the type of host cells and are known in the art, e.g. electroporation, infection with a viral vector, and the like. In the case of cancer treatment and/or prevention, the host cells may be allogenic, e.g. allogenic T cells, more preferably are autologous, in particular autologous T cells. Also, the host cells may be amplified before (re) administration to the subject, e.g. by in vitro culture; and/or may be activated, e.g. by co-culture with cells presenting the cognate peptide and/or cytokine treatment.

The term “subject”, as referred to herein, relates to a vertebrate animal, preferably a mammal, in particular a livestock, companion, or laboratory animal. Most preferably, subject is a human. Preferably, the subject has been diagnosed to suffer from cancer, is suspected to suffer from cancer, or is known to be at risk of suffering from cancer, in particular a hereditary form of cancer. Preferably, the cancer of said subject has been identified to be susceptible to treatment with a binding polypeptide, preferably as specified herein below.

The terms “medicament” and “pharmaceutical composition” are used essentially interchangeably herein and are, in principle, known to the skilled person. As referred to herein, the terms relate to any composition of matter comprising the specified active agent(s) as pharmaceutically active compound(s) and one or more excipient. The pharmaceutically active compound(s) can be present in liquid or dry, e.g. lyophilized, form. It will be appreciated that the form and character of the pharmaceutical acceptable excipient, e.g. carrier or diluent, is dictated by the amount of active ingredient with which it is to be combined, the route of administration, and other well-known variables. The excipient(s) must be acceptable in the sense of being compatible with the other ingredients of the formulation and being not deleterious to the recipient thereof. The excipient employed may include a solid, a gel, or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, physiological saline, Ringer's solutions, dextrose solution, and Hank's solution, syrup, oil, water, emulsions, various types of wetting agents, and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pennsylvania. The excipient(s) is/are selected so as not to affect the biological activity of the combination. The excipient may, however, also be selected to improve uptake of the active agent into a host cell, in particular a target cell. Thus, the excipient may also be a viral particle and/or a lipid vesicle, preferably a viral particle and/or a lipid vesicle known to mediate entry and/or fuse with the target cell of interest.

The medicament is, preferably, administered by a route as specified herein above. A therapeutically effective dose refers to an amount of the effector polypeptide or expression construct encoding the same to be used in a medicament, which prevents, ameliorates or cures the symptoms accompanying a disease or condition referred to in this specification. Therapeutic efficacy and toxicity of a drug can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. The dosage regimen will be determined by the attending physician and by clinical factors. As is well known in the medical arts, dosages for any one patient depends upon many factors, including the patient's size, age, the particular formulation of the medicament to be administered, sex, time and route of administration, general health, and other drugs being administered concurrently. The medicament referred to herein is, preferably, administered at least once, e.g. as a bolus. However, the medicament may be administered more than one time and, preferably, at least twice, e.g. permanently or periodically after defined time windows. Progress can be monitored by periodic assessment. Dosage recommendations may be indicated in the prescriber or user instructions in order to anticipate dose adjustments depending on the considered recipient.

The medicament according to the present invention may comprise further active agents in addition to the aforementioned active agent(s). Preferably, the pharmaceutically active compound as specified herein is to be applied together with at least one further drug and, thus, may be formulated together with this at least one further drug as a medicament. More preferably, in case of cancer treatment, said at least one further active agent is a chemotherapeutic agent or a further immunotherapeutic agent, such as an immune checkpoint modulator. Also, it is to be understood that the formulation of a pharmaceutical composition preferably takes place under GMP standardized conditions or the like in order to ensure quality, pharmaceutical safety, and effectiveness of the medicament.

The present invention also relates to a method of identifying a target cell expressing Protein Tyrosine Phosphatase Receptor Type Z1 (PTPRZ1) and/or Neuroligin-4, X-linked (NLGN4X), comprising
(i) contacting said target cell with a binding polypeptide of the present invention;
(ii) detecting binding of the binding polypeptide to said target cell; and
(iii) based on the detecting in step (ii), identifying a target cell expressing PTPRZ1 and/or NLGN4X.

The method of identifying a target cell expressing PTPRZ1 and/or NLGN4X, preferably, is an in vitro method. The method may in particular be performed on a cultured cell or a sample from a subject, preferably is performed on a sample from a subject, preferably a sample comprising or suspected to comprise cancer cells, preferably a tumor sample. In accordance, the target cell may be, but does not necessarily have to be, a host cell. Preferably, the term “target cell”, as used herein, relates to a vertebrate cell, more preferably a mammalian cell, even more preferably a primate cell, most preferably a human cell. The target cell may, however also be a cell of a laboratory, livestock, or companion animal, such as a mouse cell, a rat cell, a guinea pig cell, a cow cell, a sheep cell, a pig cell, a dog cell, or a cat cell. Preferably, the target cell is known or suspected to be a cancer cell, preferably is a cell comprised in a tumor.

Binding of the binding polypeptide in step (ii) may be detected by any method deemed appropriate by the skilled person, e.g. by immunological methods. Preferably, the binding polypeptide comprises a detectable label, such as a dye or an enzyme catalyzing a detectable chemical reaction, in such case. As the skilled person will understand in view of the description herein, detection of binding of the binding polypeptide in step (ii) is indicative of a target cell expressing PTPRZ1 and/or NLGN4X in step (iii). As will also be understood by the skilled person, the detecting in step (ii) may be qualitative, preferably is semiquantitative or quantitative.

The present invention also relates to a method of identifying a cancer susceptible to treatment with a binding polypeptide of the present invention, a polynucleotide of the present invention, a vector of the present invention, and/or a host cell of the present invention, comprising (I) identifying target cells expressing PTPRZ1 and/or NLGN4X in a cancer sample; and (II) identifying a cancer susceptible to treatment based on the identifying in step (I).

The method of identifying a cancer susceptible to treatment, preferably, is an in vitro method, preferably performed on a cancer sample. The term “sample”, as used herein, refers to a sample comprising cells of a subject; the sample may be a sample of a bodily fluid, preferably a sample of blood, saliva, sputum, urine, or a sample comprising separated cells or to a sample from a tissue or an organ. Separated cells may be obtained from the body fluids, such as lymph, blood, plasma, serum, liquor and other, or from the tissues or organs by separating techniques such as centrifugation or cell sorting. More preferably, the sample is a sample known or suspected to comprise cancer cells. Preferably, the sample is a cancer sample, the term “cancer sample” preferably relating to a sample known or suspected to comprise target cells, preferably cancer cells; thus, the sample preferably is a sample of a primary tumor, an infiltrate, a metastasis, a lymph node, or a relapse. The cancer sample may, however, also be a sample of single cells, e.g. in case of non-solid tumors. The sample can be obtained from the subject by routine techniques, which are well known to the person skilled in the art, e.g., venous or arterial puncture or open biopsy including aspiration of tissue or cellular material from a subject. For those areas which cannot be easily reached via an open biopsy, a surgery and, preferably, minimal invasive surgery can be performed.

As is understood by the skilled person in view of the description provided herein, a cancer susceptible to treatment is preferably identified in step (II) in case target cells expressing PTPRZ1 and/or NLGN4X are identified in the cancer sample in step (I). Preferably, a cancer susceptible to treatment is identified in case the fraction of target cells expressing PTPRZ1 and/or NLGN4X in the cancer sample is at least 10%, preferably at least 20%, more preferably at least 30%, even more preferably at least 40%, most preferably at least 50%. Also preferably, cells in the cancer sample are identified to express PTPRZ1 and/or NLGN4X in case the binding of the binding polypeptide is increased, preferably significantly increased, compared to a control, e.g. of cells known to not express PTPRZ1 and/or NLGN4X.

The present invention also relates to a use of a binding polypeptide of the present invention, a polynucleotide of the present invention, a vector of the present invention, and/or a host cell of the present invention in the manufacture of a composition for diagnosing, treating, and or preventing disease, preferably cancer.

The present invention further relates to a use, preferably in vitro use, of a binding polypeptide of the present invention, a polynucleotide of the present invention, a vector of the present invention, and/or a host cell of the present invention, for detecting a target cell expressing Neuroligin-4, X-linked (NLGN4X) and/or Protein Tyrosine Phosphatase Receptor Type Z1 (PTPRZ1).

Furthermore, the present invention relates to a kit comprising a binding polypeptide of the present invention, a polynucleotide of the present invention, a vector of the present invention, and/or a host cell of the present invention, comprised in a housing and/or further comprising a means of administration.

The term “kit”, as used herein, refers to a collection of the aforementioned compounds, means or reagents, which may or may not be packaged together. The components of the kit may be comprised by separate vials (i.e. as a kit of separate parts) or provided in a single vial, e.g. as a composition as specified herein above. The housing of the kit in an embodiment allows translocation of the compounds of the kit, in particular common translocation; thus, the housing may in particular be a transportable container comprising all specified components. Moreover, it is to be understood that the kit of the present invention may be used for practicing the methods referred to herein above. It is, preferably, envisaged that all components are provided in a ready-to-use manner for practicing the methods referred to above. Further, the kit preferably contains instructions for carrying out said methods. The instructions can be provided by a user's manual on paper or in electronic form. For example, the manual may comprise instructions for interpreting the results obtained when carrying out the aforementioned methods using the kit. Preferably, the kit comprises further compounds, such as a reaction buffer, a hybridization solution, a lysis buffer, or the like. Preferably, the kit is adapted for use in a method of the present invention, more preferably is adapted to comprise all reagents required to perform said method or methods.

Means of administration may include a delivery unit for the administration of the compound or composition and a storage unit for storing said compound or composition until administration. However, it is also contemplated that the means of administration may appear as separate devices in such an embodiment and are, preferably, packaged together in said kit. Preferred means for administration are those, which can be applied without the particular knowledge of a specialized technician. In a preferred embodiment, the means for administration is a syringe, more preferably with a needle, comprising the compound or composition as specified. In another preferred embodiment, the means for administration is an intravenous infusion (IV) equipment comprising the compound or composition. In still another preferred embodiment, the means for administration is an inhaler comprising the compound of the present invention, wherein, more preferably, said compound is formulated for administration as an aerosol.

The present invention also relates to a device comprising a binding polypeptide of the present invention, a polynucleotide of the present invention, a vector of the present invention, and/or a host cell of the present invention.

As used herein, the term “device” includes any and all contraptions comprising the components specified. Preferably, the device is a means of administration as specified herein above. Preferably, the device is adapted to perform a method as specified herein, in particular a diagnostic method and/or a method of identification. Thus, the device may also be a diagnostic device. Thus, the device preferably comprises (i) an analysis unit comprising a means for determining binding of a binding polypeptide to a target cell, and, operatively connected thereto (ii) an evaluation unit comprising tangibly embedded executable instructions for performing a method as specified herein. Typical means for determining binding of a binding polypeptide to a target cell, and means for carrying out the determination are known to the skilled person. As is understood by the skilled person, means for determining binding of a binding polypeptide to a target cell include means capable for determining an amount of binding polypeptide bound to a target cell, such as an ELISA reader, as well as means for determining effects of binding of a binding polypeptide to a target cell, such as an optical unit detecting a signal of a reporter gene assay. An evaluation means is any means capable of providing the analysis as specified; preferably, the evaluation means is a data processing means, such as a microprocessor, a handheld device such as a mobile phone, or a computer. How to link the means in an operating manner will depend on the type of means included into the device. In an embodiment, the means are comprised by a single device. Said device may accordingly include (i) an analyzing unit for the measurement of binding of a binding polypeptide to a target cell and a (ii) computer unit for processing the resulting data for the evaluation. Preferably, the instructions and interpretations are comprised in an executable program code comprised in the device, such that, as a result of determination a classification of the subject as amenable to therapy. Typical devices are those, which can be applied without the particular knowledge of a specialized technician, e.g., test stripes or electronic devices, which merely require loading with a sample. The results may be given as output of raw data, which need interpretation by a technician. Preferably, the output of the device is, however, processed, i.e. evaluated, raw data, the interpretation of which does not require a technician. Further typical devices comprise the analyzing units/devices (e.g., biosensors, arrays, solid supports coupled to ligands specifically recognizing binding, Plasmon surface resonance devices, NMR spectrometers, mass-spectrometers etc.) or evaluation units/devices referred to above in accordance with the method of the invention. Preferably, the device further comprises a memory unit, preferably comprising a database comprising at least one reference value for a binding of a binding polypeptide to a target cell.

In view of the above, the following embodiments are particularly envisaged:

Embodiment 1: A binding polypeptide comprising a first variable T cell receptor (TCR) domain and a second variable TCR domain.

Embodiment 2: The binding polypeptide of embodiment 1, (i) wherein the complementarity determining region 3 (CDR3) of the first variable TCR domain comprises, preferably consists of, the amino acid sequence of SEQ ID NO:1 or a sequence at least 80% identical thereto; and/or wherein the CDR3 of the second variable TCR domain comprises, preferably consists of, the amino acid sequence of SEQ ID NO:2 or a sequence at least 80% identical thereto; or (ii) wherein the CDR3 of the first variable TCR domain comprises, preferably consists of, the amino acid sequence of SEQ ID NO:3 or a sequence at least 80% identical thereto; and/or wherein the CDR3 of the second variable TCR domain comprises, preferably consists of, the amino acid sequence of SEQ ID NO:4 or a sequence at least 80% identical thereto.

Embodiment 3: The binding polypeptide of embodiment 1 or 2, wherein said first variable TCR domain is a TCR alpha variable domain.

Embodiment 4: The binding polypeptide of any one of embodiments 1 to 3, wherein said second variable TCR domain is a TCR beta variable domain.

Embodiment 5: The binding polypeptide of any one of embodiments 1 to 4, wherein said first variable TCR domain comprises, preferably consists of, the amino acid sequence of SEQ ID NO: 5 or an amino acid sequence at least 70% identical to SEQ ID NO:5 and/or wherein said second variable TCR domain comprises, preferably consists of, the amino acid sequence of SEQ ID NO:6 or an amino acid sequence at least 70% identical to SEQ ID NO:6.

Embodiment 6: The binding polypeptide of any one of embodiments 1 to 5, wherein said first variable TCR domain comprises, preferably consists of, the amino acid sequence of SEQ ID NO: 7 or an amino acid sequence at least 70% identical to SEQ ID NO:7 and/or wherein said second variable TCR domain comprises, preferably consists of, the amino acid sequence of SEQ ID NO:8 or an amino acid sequence at least 70% identical to SEQ ID NO:8.

Embodiment 7: The binding polypeptide of any one of embodiments 1 to 6, wherein said first variable TCR domain comprises, preferably consists of, the amino acid sequence of SEQ ID NO: 5 and/or wherein said second variable TCR domain comprises, preferably consists of, the amino acid sequence of SEQ ID NO:6.

Embodiment 8: The binding polypeptide of any one of embodiments 1 to 7, wherein said first variable TCR domain comprises, preferably consists of, the amino acid sequence of SEQ ID NO: 7 and/or wherein said second variable TCR domain comprises, preferably consists of, the amino acid sequence of SEQ ID NO:8.

Embodiment 9: The binding polypeptide of any one of embodiments 1 to 8, wherein said amino acid sequence of SEQ ID NO:5 is encoded by the nucleic acid sequence of SEQ ID NO: 9.

Embodiment 10: The binding polypeptide of any one of embodiments 1 to 9, wherein said amino acid sequence of SEQ ID NO:6 is encoded by the nucleic acid sequence of SEQ ID NO: 10.

Embodiment 11: The binding polypeptide of any one of embodiments 1 to 10, wherein said amino acid sequence of SEQ ID NO:7 is encoded by the nucleic acid sequence of SEQ ID NO: 11.

Embodiment 12: The binding polypeptide of any one of embodiments 1 to 11, wherein said amino acid sequence of SEQ ID NO:8 is encoded by the nucleic acid sequence of SEQ ID NO: 12.

Embodiment 13: The binding polypeptide of any one of embodiments 1 to 12, wherein said first variable TCR domain and said second variable TCR domain are comprised in a fusion polypeptide.

Embodiment 14: The binding polypeptide of any one of embodiments 1 to 13, wherein said first variable TCR domain is covalently connected to a first constant TCR domain, and/or wherein said second variable TCR domain is covalently connected to a second constant TCR domain.

Embodiment 15: The binding polypeptide of any one of embodiments 1 to 14, wherein said binding polypeptide recognizes its cognate peptide when presented on an MHC class I molecule, more preferably an HLA-A molecule, more preferably an HLA-A*02 molecule.

Embodiment 16: The binding polypeptide of any one of embodiments 1 to 15, wherein said binding polypeptide recognizes a peptide having the amino acid sequence NLDTLMTYV (SEQ ID NO:13), preferably when presented on an MHC class I molecule, more preferably an HLA-A molecule, more preferably an HLA-A*02 molecule.

Embodiment 17: The binding polypeptide of any one of embodiments 1 to 16, wherein said binding polypeptide recognizes a peptide having the amino acid sequence MIWEHNVEV (SEQ ID NO:14), preferably when presented on an MHC class I molecule, more preferably an HLA-A molecule, more preferably an HLA-A*02 molecule.

Embodiment 18: The binding polypeptide of any one of embodiments 1 to 17, wherein said first variable TCR domain and said second variable TCR domain are covalently connected to a transmembrane domain, preferably a TCR transmembrane domain.

Embodiment 19: The binding polypeptide of any one of embodiments 1 to 18, wherein said binding polypeptide is a TCR.

Embodiment 20: The binding polypeptide of any one of embodiments 1 to 18, wherein said first variable TCR domain and said second variable TCR domain are covalently connected to a signaling domain.

Embodiment 21: The binding polypeptide of any one of embodiments 1 to 18 and 20, wherein said binding polypeptide is a chimeric antigen receptor (CAR).

Embodiment 22: The binding polypeptide of any one of embodiments 1 to 17, wherein said first variable TCR domain and said second variable TCR domain are covalently connected to an Fc domain of an immunoglobulin.

Embodiment 23: The binding polypeptide of any one of embodiments 1 to 17 and 22, wherein said binding polypeptide is a soluble TCR, preferably is a soluble TCR tetramer.

Embodiment 24: The binding polypeptide of any one of embodiments 1 to 23, wherein said binding polypeptide further comprises a label and/or an effector compound.

Embodiment 25: A polynucleotide encoding at least one of a first variable TCR domain and a second variable TCR domain of a binding polypeptide according to any one of embodiments 1 to 24.

Embodiment 26: The polynucleotide of embodiment 25, wherein said polynucleotide is an expression construct.

Embodiment 27: The polynucleotide of embodiment 25 or 26, wherein said polynucleotide is comprised in a vector, preferably in an expression vector.

Embodiment 28: A vector comprising a polynucleotide according to any one of embodiments 25 to 27.

Embodiment 29: A host cell comprising the binding polypeptide according to any one of embodiments 1 to 24, the polynucleotide according to any one of embodiments 25 to 27, and/or the vector according to embodiment 28.

Embodiment 30: The host cell of embodiment 29, wherein said host cell is a T cell, preferably a CD8+ T cell.

Embodiment 31: A binding polypeptide according to any one of embodiments 1 to 24, a polynucleotide according to any one of embodiments 25 to 27, a vector according to embodiment 28, and/or a host cell according to embodiment 29 or 30 for use in medicine.

Embodiment 32: A binding polypeptide according to any one of embodiments 1 to 24, a polynucleotide according to any one of embodiments 25 to 27, a vector according to embodiment 28, and/or a host cell according to embodiment 29 or 30 for use in treating and/or preventing cancer.

Embodiment 33: A method of treating and/or preventing cancer in a subject, said method comprising
(a) contacting said subject with a binding polypeptide according to any one of embodiments 1 to 24, the polynucleotide according to any one of embodiments 25 to 27, the vector according to embodiment 28, and/or the host cell according to embodiment 29 or 30, and
(b) thereby treating cancer in said subject.

Embodiment 34: A method of identifying a target cell expressing Protein Tyrosine Phosphatase Receptor Type Z1 (PTPRZ1) and/or Neuroligin-4, X-linked (NLGN4X), comprising
(i) contacting said target cell with a binding polypeptide according to any one of embodiments 2 to 24, preferably embodiments 22 to 24;
(ii) detecting binding of the binding polypeptide to said target cell; and
(iii) based on the detecting in step (ii), identifying a target cell expressing PTPRZ1 and/or NLGN4X.

Embodiment 35: A method of identifying a cancer susceptible to treatment with a binding polypeptide according to any one of embodiments 1 to 24, a polynucleotide according to any one of embodiments 25 to 27, a vector according to embodiment 28, and/or a host cell according to embodiment 29 or 30, comprising
(I) identifying target cells expressing PTPRZ1 and/or NLGN4X in a cancer sample; and
(II) identifying a cancer susceptible to treatment based on the identifying in step (I).

Embodiment 36: Use of a binding polypeptide according to any one of embodiments 1 to 24, a polynucleotide according to any one of embodiments 25 to 27, a vector according to embodiment 28, and/or a host cell according to embodiment 29 or 30 in the manufacture of a composition for diagnosing, treating, and/or preventing disease, preferably cancer.

Embodiment 37: Use of a binding polypeptide according to any one of embodiments 1 to 24, a polynucleotide according to any one of embodiments 25 to 27, a vector according to embodiment 28, and/or a host cell according to embodiment 29 or 30 for detecting a target cell expressing Neuroligin-4, X-linked (NLGN4X) and/or Protein Tyrosine Phosphatase Receptor Type Z1 (PTPRZ1).

Embodiment 38: The use of embodiment 37, wherein said use is an in vitro use.

Embodiment 39: A kit comprising a binding polypeptide according to any one of embodiments 1 to 24, a polynucleotide according to any one of embodiments 25 to 27, a vector according to embodiment 28, and/or a host cell according to embodiment 29 or 30, comprised in a housing and/or further comprising a means of administration.

Embodiment 40: A device comprising a binding polypeptide according to any one of embodiments 1 to 24, a polynucleotide according to any one of embodiments 25 to 27, a vector according to embodiment 28, and/or a host cell according to embodiment 29 or 30.

Embodiment 41: The subject matter of any one of embodiments 32 to 40, wherein said cancer is a brain cancer, preferably a glioma, more preferably a glioblastoma.

Embodiment 42: The subject matter of any one of embodiments 32 to 41, wherein said cancer was identified to be susceptible to treatment according to the method of embodiment 35.

All references cited in this specification are herewith incorporated by reference with respect to their entire disclosure content and the disclosure content specifically mentioned in this specification.

### FIGURE LEGENDS

FIG. 1: An HLA-A2-restricted TCR binding PTPRZ1¹⁸¹⁴⁻¹⁸²². (A) Single cell VDJ sequencing from activation-based-sorted PTPRZ1¹⁸¹⁴⁻¹⁸²²-reactive T cells following peptide vaccination from an HLA-A2-positive patient; (B) Exemplary flow cytometric analysis after transfection of TCR-deficient Jurkat T cells with the PTPRZ1¹⁸¹⁴⁻¹⁸²²-TCR containing a murine TCR beta chain for flow cytometry-based analysis of TCR expression compared to TCR beta fluorescence minus one (FMO); (C) Nuclear factor of activated T cells (NFAT) reporter based-activation assays of PTPRZ1¹⁸¹⁴⁻¹⁸²²-TCR transgenic Jurkat T cells using T2 cells as antigen presenting cells and HLA-A2-positive U87 glioma cells loaded with PTPRZ1¹⁸¹⁴⁻¹⁸²² peptide or a PTPRZ1¹⁸¹⁴⁻¹⁸²²-containing tandem minigene, respectively; (D) PTPRZ1¹⁸¹⁴⁻¹⁸²²-pentamer flow cytometry on PTPRZ1¹⁸¹⁴⁻¹⁸²² TCR-transgenic Jurkat T cells; (E) PTPRZ1¹⁸¹⁴⁻¹⁸²² killing assay using U87 and P3 as glioma target cells and healthy donor-derived T cells (n=3). Experimental conditions as indicated in the Figure. Flu is negative control peptide and TCR.

FIG. 2: An HLA-A2-restricted TCR binding NLGN4X¹³¹⁻¹³⁹. (A) Single cell VDJ sequencing from multimer-sorted NLGN4X¹³¹⁻¹³⁹-reactive T cells following peptide vaccination from an HLA-A2-positive patient; (B) Intracellular flow cytometry of NLGN4X¹³¹⁻¹³⁹-reactive TCR-transgenic healthy donor derived T cells (n=3) after overnight co-culture with peptide-loaded K562 antigen presenting cells (MOG, Myelin oligodendrocyte glycoprotein, negative control peptide; CD3/CD28, positive control); (C) NLGN4X¹³¹⁻¹³⁹-reactive TCR SFG vector-transduced primary human T cells were co-cultured with peptide-loaded U87 glioma cells as well as U87 cells overexpressing the full-length NLGN4X protein (MOG, Myelin oligodendrocyte glycoprotein, negative control peptide); (D) NLGN4X¹³¹⁻¹³⁹-reactive TCR pLEX307 vector-transduced primary human T cells were co-cultured with peptide-loaded U87 glioma cells 7 days after transduction (MOG, Myelin oligodendrocyte glycoprotein, negative control peptide); (E) Vital FR assay using NLGN4X¹³¹⁻¹³⁹-reactive TCR SFG-vector transduced TCR-transgenic primary human T cells targeting the U87 expressing NLGN4X¹³¹⁻¹³⁹-containing tandem minigene (TMG) at a E:T ratio of 2:1:

FIG. 3: NLGN4X-TCR-T showed comparable effective in vitro recognition and lysis of target cells as clinically used control TCR. (A) VDJ analysis from single cell TCR sequencing of multimer-sorted patient T cells after vaccination. Frequencies: ft1—90.82%; ft2—1.78%; ft3—0.96%; ft4—0.96%. (B) Exemplary flow cytometric analysis of the transfection efficiency of TCR-transfected Jurkat T cells. (C) NLGN4X ft1-4 TCR-transfected Jurkat76 T cells co-cultured with peptide-loaded BOLETH APC. Mean with SEM of three technical replicates. (D) NFAT-reporter assay of NLGN4X TCR transfected Jurkat76 T cells co-cultured with peptide-loaded HLA-A*02+ PBMCs. RLU=Relative luminescence units. Mean with SEM of three technical replicates. (E) NFAT-reporter assay of NLGN4X TCR transfected Jurkat76 T cells co-cultured with peptide-loaded HLA-A*02+ U87 glioma cells. RLU=Relative luminescence units. Mean with SEM of three technical replicates.

FIG. 4: Development of the manufacturing process of the T cell product. (A) Schematic overview of the TCRft1-pLEX307 EF1-alpha: EF1-alpha promoter—TCR beta chain (including the murine TCR beta constant region)—TCR alpha chain—Woodchuck Hepatitis Virus posttranscriptional regulatory element—puromycin resistance. (B) Exemplary transduction efficiency of TCRft1-pLEX307 EF1-alpha transduced human T cells by flowcytometric analysis of mTCRb, compared to Mock-transduced T cells. (C) Schematic overview of SFG-IRES-GFP vector: long terminal repeat sequence—Moloney murine leukemia virus—TCR beta chain (including the murine TCR beta constant region)—TCR alpha chain—internal ribosomal entry site—GFP—long terminal repeat sequence. (D) Exemplary transduction efficiency of TCRft1-SFG-IRES-GFP transduced human T cells by flowcytometric analysis of mTCRb, compared to Mock-transduced T cells. (E) GFP+ human T cells after transduction with the TCRft1-SFG-IRES-GFP retroviral vector. Mean with SEM. (F) mTCRb expression of primary T cells 4 days after transduction with the TCRft1-SFG-IRES-GFP retroviral vector. (G) Multi-color flow cytometry assessment of different phenotypic markers in human T cells after transduction with a TCRft1-SFG-IRES-GFP vector. n=2 biological replicates.

FIG. 5: NLGN4X-TCR-T showed comparable effective in vitro recognition and lysis of target cells as a clinically used control TCR. (A) Heatmap of the functional response (IFN-γ, TNF-α, GrzB) of three different donors transduced with a NLGN4X¹³¹⁻¹³⁹ TCR and co-cultured with peptide loaded HLA-A*02⁺ K562 leukemia cells. For statistical analysis compare FIG. 3 B-D. n=3 biological replicates. (B) TNF-α production of NLGN4X-TCR-T and Mart-1-TCR-T cultured with peptide loaded HLA-A*02+ K562 leukemia cells. Target peptide: NLGN4X-TCR-T vs. MART-1 TCR T cells p=0.0863. Mean with SEM of n=3 biological replicates. Twoway-ANOVA. (C) IFN-γ production of NLGN4X-TCR-T and Mart-1-TCR-T cultured with peptide loaded HLA-A*02+ K562 leukemia cells. Target peptide: NLGN4X-TCR-T vs. MART-1-TCR-T p=0.2926. (D) Granzyme B expression of NLGN4X-TCR-T vs. MART-1-TCR-T cultured with peptide loaded HLA-A*02+ K562 leukemia cells. Target peptide: NLGN4X-TCR-T vs. MART-1-TCR-T p=0.3461. Mean with SEM of n=3 biological replicates. Twoway-ANOVA. (E) Exemplary overview of the modified Vital-FR assay used in this study: target cells that either endogenously expressed the target epitope or were exogenously loaded with the respective peptide were labeled with CellTrace™ FarRed and non-target cells (irrelevant peptide or no target expression) were labeled with CellTrace™ Violet and cultured with target-specific TCR transduced T cells in the same well. (F) Live cells of either target peptide loaded or unloaded K562 cells after overnight co-culture with NLGN4X-TCR-T or MART-1-TCR-T assessed by flow cytometric analysis. Mean with SEM of n=3 biological replicates. Twoway-ANOVA. Target peptide for NLGN4X-TCR-T is the NLGN4X¹³¹⁻¹³⁹ peptide, for MART-1-TCR-T the MART-1 peptide (Ellingson BM et al. 2017, Rohaan M W et al. 2022, Morgan R A et al. 2013, Chheda Z S et al 2018, Immisch L et al. 2022, Kilian M et al. 2022, Bolliger MF et al. 2008, Choe JH et al. 2021, Sahillioglu AC et al. 2022). Control peptide is the MOG (Myelin oligodendrocyte glycoprotein) peptide (Sahillioglu A C et al. 2022).

FIG. 6: The NLGN4X TCR specifically recognizes and lyses glioma cells expressing the NLGN4X target epitope. (A) Heatmap of the functional response (CD69, 4.1BB, GrzB) of three different donors transduced with a NLGN4X TCR and co-cultured with peptide-loaded HLA-A*02+ U87 glioma cells. (B) Optical density (OD) measured detecting LDH release after overnight co-culture of NLGN4X-TCR-T and Flu (MHCI epitope) TCR transgenic T cells (Flu-TCR-T) with NLGN4X or Flu peptide loaded U87 glioma cells. Mean with SEM of n=3 biological replicates. Twoway-ANOVA. (C) NLGN4X-TCR-T were co-cultured with either peptide loaded U87 glioma cells or unloaded cells and specific cytotoxicity was calculated using FACS based counting of tumor cells. Mean with SEM of n=3 biological replicates. Unpaired t-Test. (D) Heatmap of the functional response (CD69, 4-1BB, GrzB) of three different donors transduced with a NLGN4X TCR or Flu TCR (here: negative control TCR) and co-cultured with U87 glioma endogenously expressing the NLGN4X protein sequence including the relevant epitope (U87 NLGN4X) or a tandem-minigene (U87 TMG) containing the antigenic sequence of NLGN4X. (E) LDH release assay of NLGN4X-TCR-T vs. Flu-TCR-T (TCR negative control) targeting either U87 NLGN4X, U87 TMG or U87 (target negative control) glioma cells. Mean with SEM of n=3 biological replicates. Twoway-RM-ANOVA. Target peptide for NLGN4X-TCR-T is the NLGN4X¹³¹⁻¹³⁹ peptide. Control peptide is the Flu (influenza)⁵⁸⁻⁶⁶ MHC class I peptide (as disclosed in Choo, J Virol, 2014), to which Flu-TCR-T are reactive (VDJdb.cdr3.net).

FIG. 7: Intracerebroventricular delivery of NLGN4X-TCR-T mediates temporary tumor regression and increased survival in an intracranial tumor model. (A) Schematic experimental overview: NSG MHC I/II KO mice were challenged with intracranial U87 NLGN4X antigen overexpressing gliomas and after confirmation of tumor growth NLGN4X-TCR-T or Flu-TCR-T were injected at day 15 and 22 after tumor inoculation. (B) Preclinical survival of mice treated either with NLGN4X-TCR-T or Flu-TCR-T targeting intracranial NLGN4X antigen overexpressing U87 cells. NTC=No T cell Control. n=9 mice for NLGN4X-TCR-T, n=8 mice for Flu-TCR-T, n=7 for NTC. Log-Rank-Test. (C) Radiographic response assessment according to the mRANO criteria: between day 11 and 67 complete response (CR) was defined as a change in tumor volume of −100%, partial response (PR) as <−65%, stable disease (SD) between >−65% and +40% and progressive disease (PD) as >+40%. (D) MRI image of one long-term surviving NLGN4X-TCR-T treated animal showing tumor regression at the initial tumor site until day 67 and tumor progression at day 98. (E) Individual growth curves of U87 NLGN4X antigen expressing glioma cells of NLGN4X-TCR-T (I) and Flu-TCR-T (II) treated animals. Circled mice were analyzed by FACS as shown in FIG. 7F. Log 10-scaled growth. Thus, tumor volumes with V=0 μl are not displayed in the graph. CR=complete response, PR=partial response, SD=stable disease, PD=progressive disease, D=death. For visualization of the tumor growth the detection limit for tumor volumes was set to 0.1 μl. (F) Representative flow cytometric analysis from two animals (M1=mouse 1, M2=mouse 2) with late-stage recurrence of the tumor showing persistence of primarily CD4+ T cells at the tumor site with a predominantly CCR7-CD45RA-effector memory phenotype and impaired proliferation with high PD-1 expression. Gated on live hCD3+ T cells. (G) Realtime quantitative PCR of the U87 TMG plasmid sequence in tumors of NLGN4X-TCR-T, Flu-TCR-T treated or NTC animals at late-stage time point compared to in vitro cultured U87 TMG and U87 cells. Relative expression to hGAPDH or hβ-Actin. Log 10-scaled. n=3 biological replicates. (H) Exemplary immunofluorescence staining of HLA-A expression: One NLGN4X-TCR-T treated animal and one untreated animal at late stage timepoint shown.

FIG. 8: NLGN4X-TCR-T exhibit an effector phenotype in the tumor microenvironment after intracerebroventricular delivery. (A) Experimental overview: U87 NLGN4X antigen overexpressing gliomas were injected intracranially and NLGN4X-TCR-T or Flu-TCR-T were injected into the contralateral ventricle. After 6 days T cells were analyzed by flow cytometry and ex vivo activation was assessed. (B) Exemplary flow cytometry plots showing intratumoral CD3+ T cells, CD4-CD8 distribution and mTCRb and GFP expression. (C) Normalized (to tumor volume) count of CD3+ CD8+ T cells in the TME. n=8 (NLGN4X-TCR-T), n=7 (Flu-TCR-T). Unpaired t-Test. (D) Exemplary FACS plots showing the CD45RA and CCR7 as well as Ki67 and PD-1 expression on intratumoral CD3+CD8+ T cells (TCM=T central memory cells, TN=naïve T cells, TEM=T effector memory cells, TEM-CD45RA+=TEM re-expressing CD45RA). (E) Heatmap of phenotypic markers of intratumoral CD3+CD8+ T cells. n=8 (NLGN4X-TCR-T), n=7 (Flu-TCR-T). (F) Assessment of activation and effector cells markers of intratumoral CD8+ T cells. n=8 (NLGN4X-TCR-T), n=7 (Flu-TCR-T). Twoway-ANOVA.

FIG. 9: Activation and cytotoxicity of PTPRZ1-specific TCR-engineered human T cells in vitro. Activation of TCR-T cell. The T cells were co-cultured with peptide-loaded end epitope-expressing U87 cells and endogenously PTPRZ1-expressing P3 cell line. The activation was assessed with flow cytometry gated on CD8+ TCR+ cells.

FIG. 10: PTPRZ1-specific TCR-engineered human T cell efficacy in vivo. NSG MHC KO mice are subcutaneously inoculated with epitope-expressing U87 cell line. The mice received 2 doses of adoptive cell transfer of transduced TCR-T cells. Tumor growth is measured by caliper.

The following Examples shall merely illustrate the invention. They shall not be construed, whatsoever, to limit the scope of the invention.

### EXAMPLE 1: MATERIALS AND METHODS

### 1.1 Single Cell TCR-Seq

Single-cell capturing and downstream library constructions of FACS-sorted cells were performed using Chromium Single Cell V(D)J Reagent kit v1 chemistry (10× Genomics; PN-1000006, PN-1000020, PN-1000005, PN-120262) according to the manufacturer's protocol. The constructed scVDJ library was sequenced on HiSeq2500 rapid platforms (Illumina).

### 1.2 Cell Lines and Modification of Gene Expression

U87 glioma cells were cultured in DMEM with FBS, Penicillin and Streptomycin. U87 NLGN4X were generated by transfection with the full-length NLGN4X protein coding sequence in pMXs-IRES-PuroR using FuGene HD transfection reagent (Promega). Prior to transfection U87 cells were seeded at density of 100.000 cells per well in a six well plate and rested for 24 h. Cells were transfected with 2 μg of the respective plasmid DNA, rested for 48 hours and then positively selected under application of 5 μg/ml Puromycin. TMG containing epitopes of PTPRZ1 (1814-1822) and NLGN4X (131-139) were cloned into pMXs-IRES-PuroR and transfected into U87 cell line as above but selected with 2 μg/ml Puromycin.

### 1.3 Cloning of Human TCR Constructs

The variable chains of the TCR were synthesized by Eurofins and cloned into an S/MAR vector (pSMARTer V8) containing the murine constant alpha and beta chains using the NEB Golden Gate assembly mix (New England Biolabs). This construct was used for electroporation of TCR-deficient Jurkat T cells. Interventional and control TCRs were cloned into an SFG vector generated using In-Fusion cloning from Takara Bio Europe.

### 1.4 Electroporation of Jurkat T Cells

2 Mio cells of Jurkat CD8+ TCR deficient T cells per electroporation were used. 5 μg of TCR encoding vector was used respectively and an NFAT reporter encoding vector was additionally delivered to Jurkats. (settings: 1325 V, 3 pulses, 10 ms, Neon transfection system). After 24 hours incubation TCR expression was determined by flow cytometric analysis of TCRbeta positive cells compared to untransfected T cells.

### 1.5 Isolation of Human T Cells

Whole blood samples of healthy donors were obtained from the IKTZ Heidelberg and PBMCs were isolated by density gradient-based centrifugation with Lympho-Paque solution (Genaxxon). Subsequently, obtained PBMCs were washed with PBS supplemented with 1 mM EDTA and finally frozen down for later downstream application. Human T cells were isolated from healthy donor PBMCs using MagniSort™ Human T cell Enrichment Kit (Invitrogen) or Pan T Cell Isolation Kit, human (Miltenyi).

### 1.6 Lentiviral Transduction of Primary Human T Cells

HEK 293 T cells were co-transfected with a TCR encoding lentiviral construct (pLEX307) and Ready-to-Use Lentiviral Packaging Plasmid Mix (Cellecta) using Fugene transfection reagent. Virus containing supernatant was harvested after 24 h, 48 h and 72 h. T cells were directly activated after isolation from fresh peripheral blood mononuclear cells and activated for 24 hours in Xvivo medium supplemented with human Interleukin 7 (0.2 IU/ml), human Interleukin (290 IU/ml) and 10 yl per 1 million T cells human T Cell TransAct™ beads (Miltenyi). Upon transduction lentiviral supernatant was spin-loaded onto Retronectin-coated 6 well-plates. Subsequently, up to 2 Mio. activated T cells/well were transduced by spinoculation on Virus loaded plates and incubated overnight. TCR expression was assessed after 48 h.

### 1.7 Retroviral Transduction of Primary Human T Cells

In brief, T cells were directly activated after isolation from fresh PBMCs and activated for 48 hours in CTL medium (45% RPMI, 45% Click's medium, 10% FBS supplemented with 10 ng/ml hIL-7 and 5 ng/ml hIL-15) and Transact Beads (Miltenyi) according to manufacturer's instructions.

HEK 293T cells were seeded at a density of 3 Mio cells per 10 cm dish and co-transfected with the TCR encoding SFG vector and RD-114 and PeqPam as helper plasmids using Fugene HD transfection reagent. Viral supernatant was harvested after 48 hours, filtered and spinloaded onto previously Retronectin coated 24 well plates. Subsequently, viral supernatant was discarded and 0.5Mio T cells were added in 1 ml of CTL medium and incubated for 4 days at 37° C., 5% CO₂. TCR transduction efficiency was assessed by flowcytometric analysis of the GFP and TCRbeta expression in CD3 T cells.

### 1.8 NFAT Reporter-Based Activation Assays

Jurkat TCR deficient T cells were equipped with the respective TCR and NFAT reporter by electroporation as indicated previously. U87 glioma cells or T2 cells at a density of 3 Mio cells/ml were peptide pulsed for 1 hour or overnight at a final peptide concentration of 10 μg/ml in 96 well plates. After confirmation of TCR expression Jurkat T cells at a density of 3 Mio cells/ml were co-cultured with peptide loaded antigen presenting cells overnight. Subsequently, Nano-Glo Luciferase assay reagents (Promega) were diluted according to the manufacturer's instruction and added to the co-culture. Luminescence was recorded on a PHERAstar FS plate reader and analysed using MARS Data analysis software (BMG Labtech). Activation was assessed by NFAT reporter assay or a flow cytometric assessment of cluster of differentiation (CD) 69 Expression for Jurkat Cells Co-Cultures

### 1.9 Human T Cell Activation and Cytotoxicity Assays

T cells were isolated and activated as above and the respective TCRs were delivered by retroviral transduction. 4 days post transduction, TCR-T cells were used for in vitro T cell activation and cytotoxicity assays using Peptide loaded U87 glioma cells or K562 cells, U87 TMG-expressing glioma cells or NLGN4X overexpressing U87 glioma cells as target cells and incubated overnight. Subsequently, cytotoxicity was measured using a CytoTox 96® Non-Radioactive Cytotoxicity Assay (Promega, see below) and T cells were stained extra-and intracellularly for flow cytometric analysis of TNF-alpha, IFN-y and Granzyme B expression.

### Vital-FR Assay

T cell receptor transduced human T cells were used on day 4 after transduction and co-cultured overnight at 2:1 effector to target cell ratio with different target cell lines previously labeled with CellTrace reagents according to manufacturer's instructions at a dilution of 1:5000. After overnight co-culture plates were centrifuged and resuspended in PBS. T cells were discarded and 30 μl Trypsin per well was added and after 10 min tumor cells were resuspended and subjected to extracellular FACS staining using eFluor780 conjugated fixable viability dye (1:1000 eBioscience) and PE-conjugated anti-human CD3 (1:100, BioLegend) for negative selection of tumor cells.

Vital-FR cytotoxicity assay and flow cytometry-based cytotoxicity assays

TCR-transduced human T cells were used on day 4 after transduction and co-cultured overnight at different effector-to-target (E:T) cell ratios with different target cell lines that had been previously labeled with CellTrace™ Far Red or Violet reagents according to manufacturer's instructions at 0.2 μM. If peptide pulsed target cells were used as antigen presenting cells (APC), APC were loaded at the indicated peptide dilution for 1 hour at 37° C., 5% CO₂ prior to the assay. Following overnight co-culture, plates were centrifuged and resuspended in PBS. T cells were removed and 30 μl trypsin per well was added and after 10 min tumor cells were resuspended and subjected to flow cytometry using eFluor780-conjugated fixable viability dye and PE-conjugated anti-human CD3 for negative selection of tumor cells. 123 Counting beads were added in 50 μl at a 1:5 dilution. Cytotoxicity was calculated using normalized count of tumor cells. Cytotoxicity (in %) was calculated as the quotient of live tumor cells after T cell co-culture to either a tumor cell only or unloaded tumor cell control.

### LDH Cytotoxicity Assay

After 24 hours co-cultures were centrifuged and 50 μl of the supernatant was used and incubated with CytoTox 96 Reagent (Promega) for 30 minutes in the dark at room temperature. After 30 min, stop solution was added and absorbance at 490 nm was recorded. Minimum LDH release of T cells and Target cells and media background signal were measured for subsequent analysis.

### 1.10 Flow Cytometry

For intracellular staining cells were incubated with 5 μg per ml Brefeldin A (Sigma-Aldrich) for 5 hours at 37° C., 5% CO₂ to allow intracellular enrichment of targeted proteins. For human T cells, PBS supplemented with 10% human serum was used for blocking, and cells were subsequently stained. For intracellular staining of cytokines cells were fixed and permeabilized using IC fixation buffer and IC permeabilization buffer, respectively (both eBioscience) and then stained with the relevant antibodies for 45 minutes at 4° C. Human T cells were stained in 50 μl or 100 μl with an antibody dilution of 1:50.

### 1.11 Mice

NOD Cg-Prkdc^{scid} H2-K1^{tm1Bpe} H2-Ab^{1em1Mvw} H2-D1^{tm1Bpe} Il2rg^{tm1Wjl}/SzJ (NSG MHCI/II KO) mice were kindly provided by L. D. Shultz and bred at the DKFZ animal facility. All animal procedures were performed following the institutional laboratory animal research guidelines and were approved by the governmental institutions (Regional Administrative Authority Karlsruhe, Germany, file number: G-37/18). For experimental groups 6-to 28-weeks old mice were matched by age and sex.

### 1.12 Intracranial Tumor Experiments and Intracerebroventricular Injection of T Cells

For orthotopic tumor cell injection, 1×10⁵ U87 tandem-minigene (TMG) glioma cells were injected at a concentration of 50×10⁶ cells per ml in 2 μl PBS into the right hemisphere of NSG MHCI/II KO mice 1 mm anterior to the coronal suture, 2 mm lateral to the bregma and in 3 mm depth. T cells were transduced and expanded as described above. After confirmation of tumor growth using magnet resonance imaging (MRI), up to 5×10⁶ T cells were injected into the left lateral ventricle at 0.5 mm lateral to the bregma and 2.2 mm depth of tumor bearing mice in a total volume of 4 μl PBS. For survival experiments, T cells were injected on day 15 and 22, for assessment of intratumoral phenotype one injection on day 48 was performed. Tumor growth was monitored with repetitive MRI and mice were checked daily for tumor-related symptoms and sacrificed when the humane or experimental stop criteria (Regional commission Karlsruhe, file number: G-37/18) were met. Obtained tissue was either directly processed for flow cytometry or cryo-fixed and assessed by immunofluorescence.

### 1.13 Statistical Analysis

All results were analyzed with Prism version 9.4.0. Statistical tests are indicated in the respective figure legends. ANOVA tests were corrected for multiple testing using Sidak's multiple comparisons correction. Results were considered as significant if the p-value was below 0.05.

### EXAMPLE 2: RESULTS

### 2.1 Results on PTPRZ1-Specific TCR

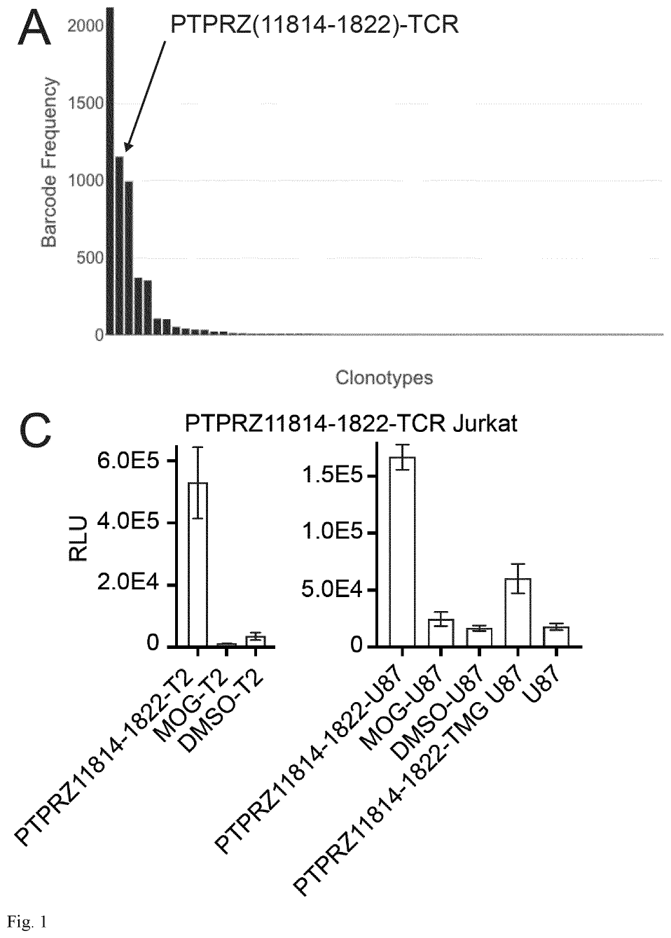

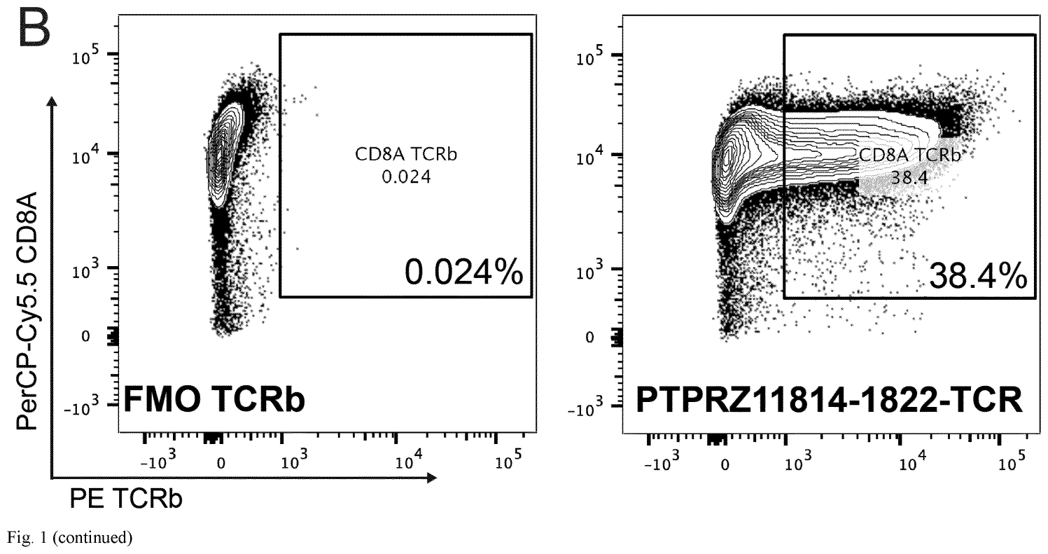

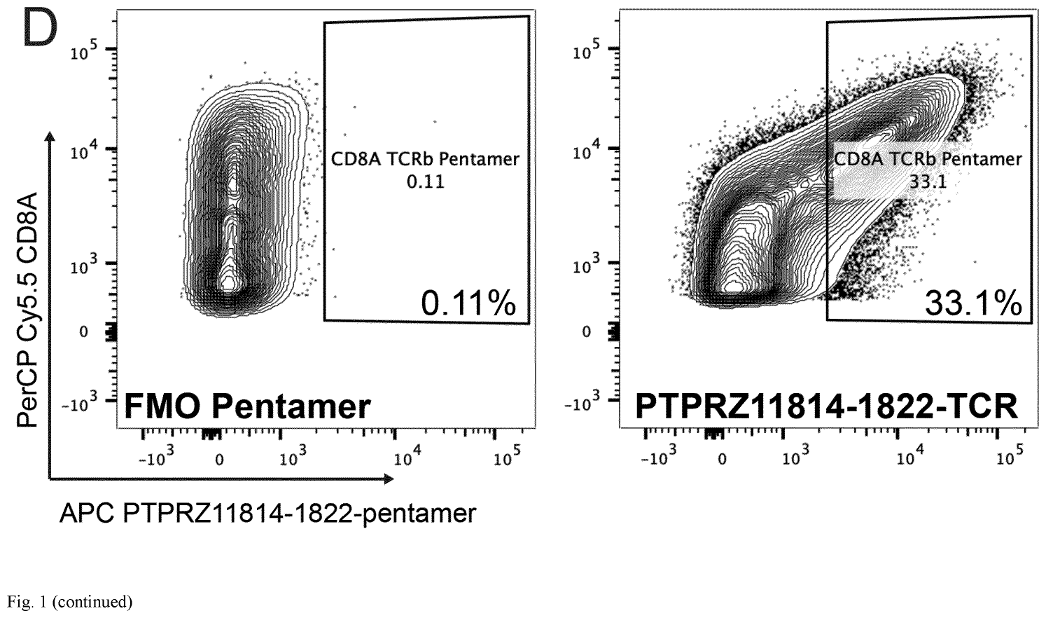

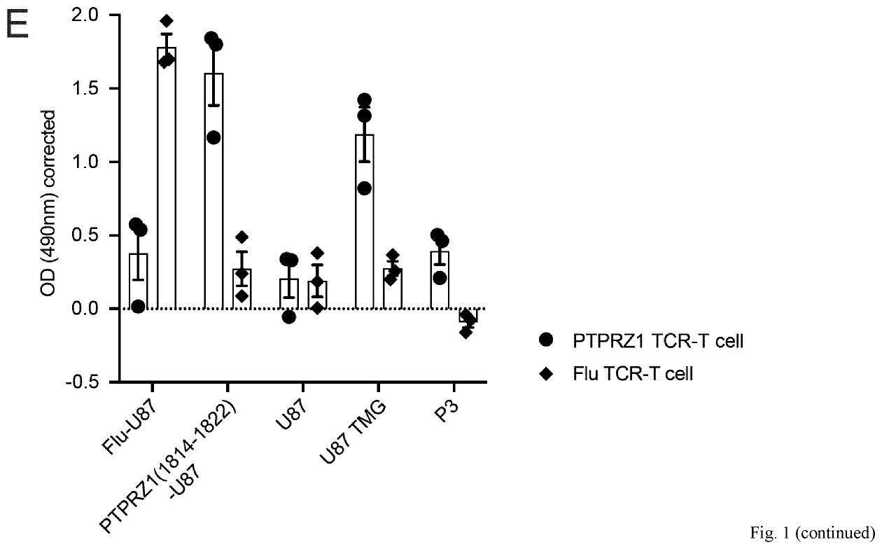

The PTPRZ1(1814-1822)-specific TCR was identified by single cell sequencing from a patient that had received a PTPRZ1(1814-1822)-peptide containing multi-peptide vaccine. Single cell TCR sequencing following activation-based enrichment of T cells revealed an oligoclonal T cell population (FIG. 1A). The subsequently identified PTPRZ1(1814-1822) TCR harboring a murine TCR beta constant chain was overexpressed in Jurkat T cells as demonstrated by murine TCRB cell surface expression (FIG. 1B). Both, HLA-A*02:01-positive and PTPRZ1(1814-1822) peptide-loaded T2 as wells as glioma cells U87, specifically activated PTPRZ1(1814-1822) TCR-transgenic Jurkat T cells in a nuclear factor of activated T cells (NFAT) reporter based-activation assay (FIG. 1C). In addition, expressing a PTPRZ1(1814-1822)-containing tandem minigene (TMG) in U87 leads to robust activation of PTPRZ1(1814-1822) TCR-transgenic Jurkat T cells. Moreover, these PTPRZ1(1814-1822) TCR-transgenic Jurkat T cells could be identified by PTPRZ1(1814-1822)-pentamer flow cytometry (FIG. 1D). Most importantly, healthy donor-derived T cells that were stably overexpressing the PTPRZ1(1814-1822) TCR robustly killed PTPRZ1(1814-1822)-loaded U87 as wells as P3, an HLA-A*02:01-positive patient-derived primary glioblastoma cell line with endogenous PTPRZ1 expression (FIG. 1E).

### 2.2 Results on NLGN4X-Specific TCR

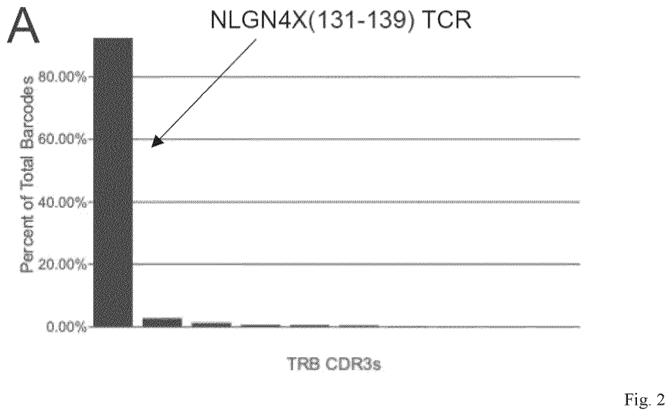

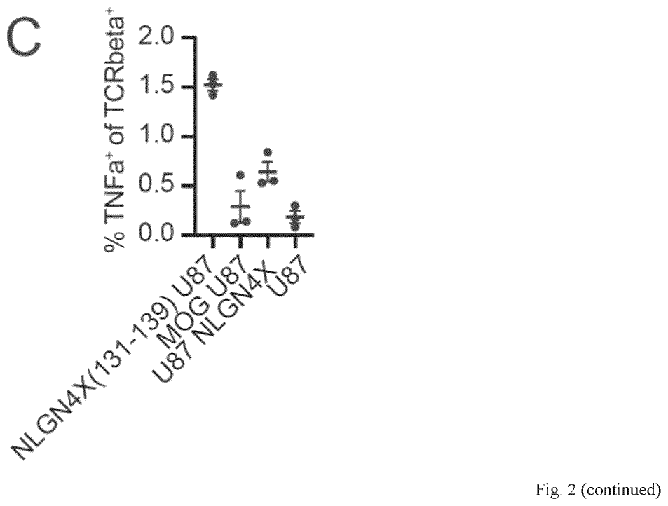

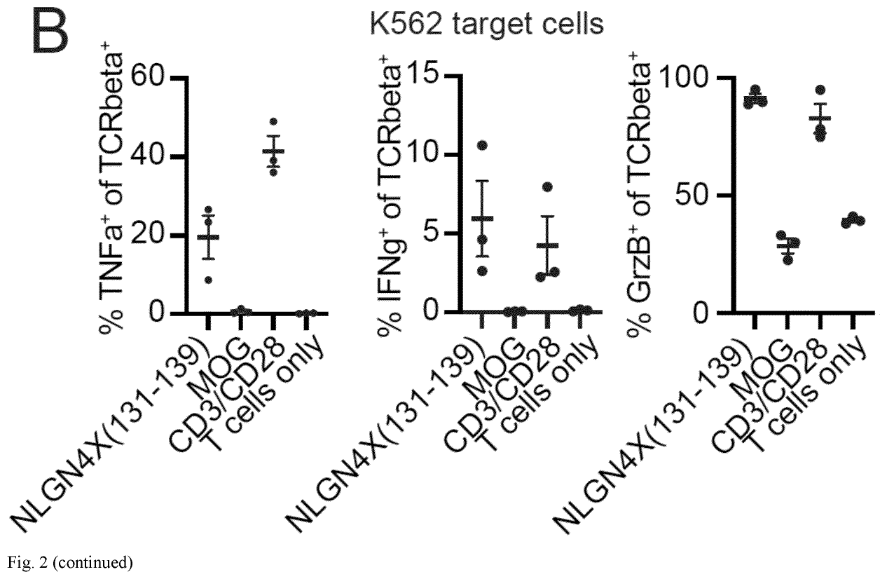

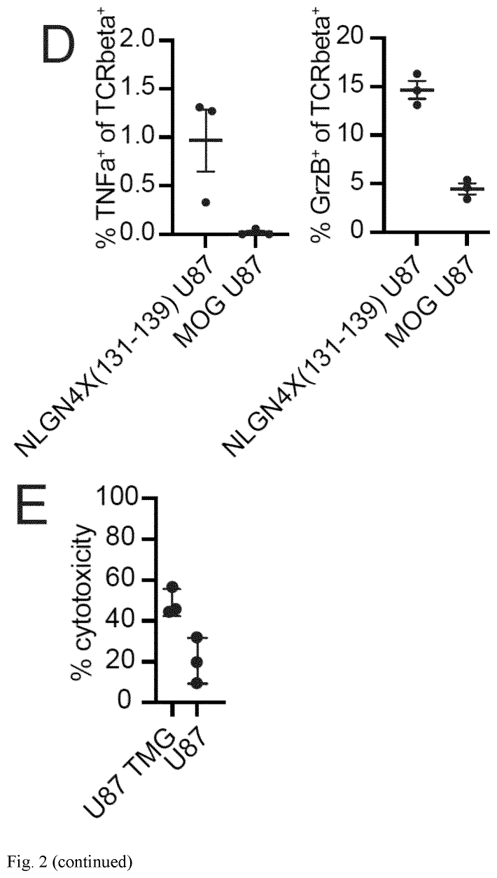

The NLGN4X(131-139)-specific TCR was identified by single cell sequencing from a patient that had received an NLGN4X(131-139)-peptide containing multi-peptide vaccine. Single cell TCR sequencing following multimer-based enrichment of T cells revealed a monoclonal T cell population (FIG. 2A). Using an NLGN4X(131-139) peptide-loaded HLA-A2-positive K562 cell line, specifically activated NLGN4X(131-139) TCR-transgenic primary healthy donor T cells leading to polyfunctional cytokine production upon target recognition assessed by intracellular flow cytometry (FIG. 2B). Importantly, U87 glioma cells loaded with the NLGN4X(131-139) peptide or expressing a full-length NLGN4X lead to robust polyfunctional cytokine production by NLGN4X(131-139) TCR-transgenic primary healthy donor T cells (FIG. 2C, D). Most importantly, healthy donor-derived T cells that were stably overexpressing the NLGN4X(131-139) TCR robustly killed NLGN4X(131-139)-containing tandem mini gene expressing U87 glioma cells (FIG. 2E).

### 2.3 NLGN4X¹³¹⁻¹³⁹-Reactive TCR Identified by Single Cell TCR-Seq From a Vaccinated Glioblastoma Patient

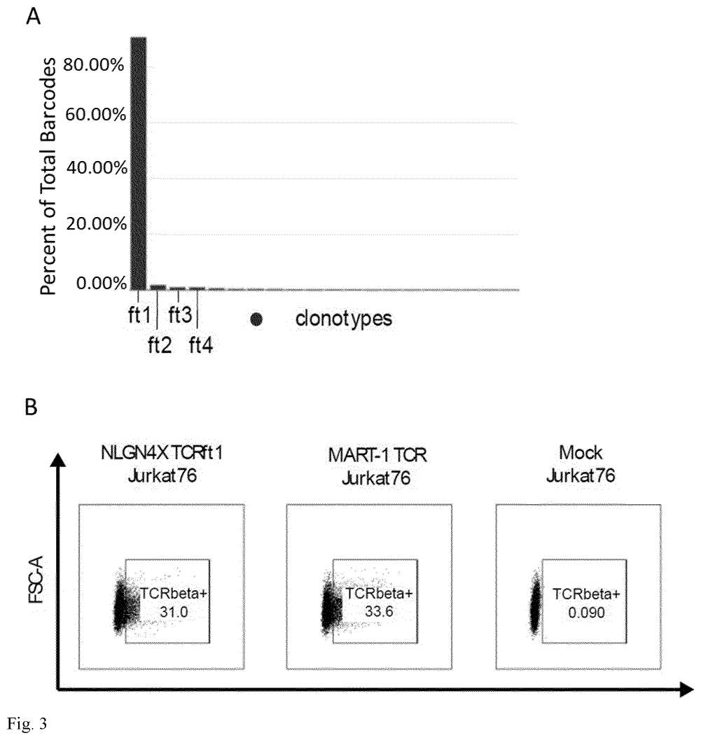

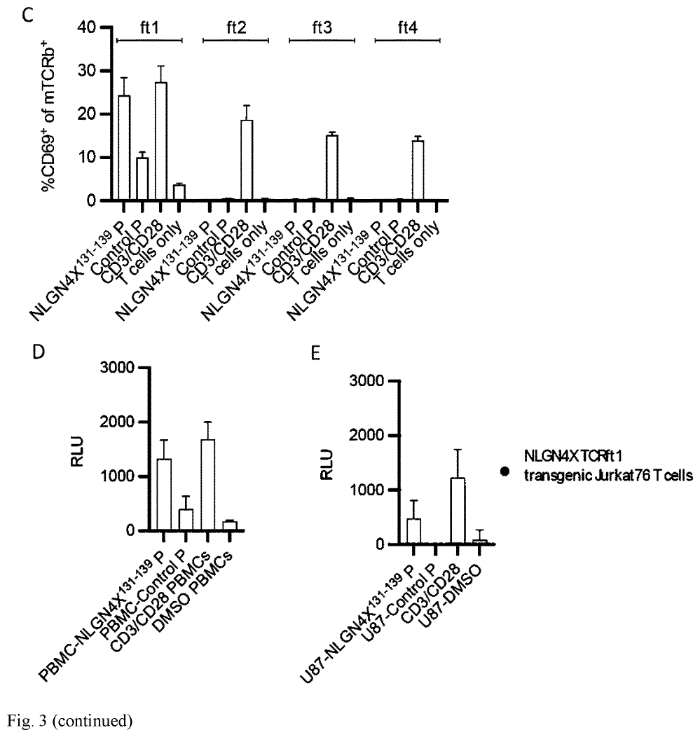

NLGN4X¹³¹⁻¹³⁹-HLA-A*02⁺ tetramer-enriched CD45⁺ CD3⁺ T cells from a patient with expansion of NLGN4X¹³¹⁻¹³⁹-reactive T cells following administration of a multi-peptide vaccine including NLGN4X¹³¹⁻¹³⁹ were subjected to single cell VDJ sequencing (scVDJ-seq). The tetramer-enriched T cell pool was highly clonal and one single clonotype made up 90.82% of the TCR repertoire (FIG. 3A). Next, episomal nano scaffold matrix attachment region (nano-S/MAR) DNA vectors with the respective variable alpha and beta chains of the top 4 TCR clones (frequotype 1-4=ft1-4) and transfected TCR-deficient Jurkat T cells (Jurkat76) were generated. TCR surface expression was confirmed by flow cytometric analysis of the murine TCR beta chain (mTCRb) allowing specific detection of the transgenic TCR and excluding mispairing with the endogenous TCR (FIG. 3B). TCR-transfected Jurkat76 were then co-cultured with peptide-loaded BOLETH HLA-A*02⁺ presenter cells and CD69 expression was assessed by flow cytometry. The T cell activation marker CD69 was upregulated only in TCRft1-expressing Jurkat76 when exposed to the NLGN4X¹³¹⁻¹³⁹-epitope (FIG. 3C). Thus, TCRft1 was consequently used for downstream reactivity assessment. As mode of action for antigen-dependent T cell stimulation, nuclear translocation of nuclear factor of activated T cells (NFAT) represents a hallmark of TCR signaling. Therefore, TCRft1-expressing Jurkat76 were co-transfected with a luciferase NFAT-reporter and subsequently co-cultured with peptide-loaded HLA-A*02⁺ peripheral blood mononuclear cells (PBMC) and HLA-A*02⁺ U87 glioma cells (FIG. 3D, E) that do not endogenously express the NLGN4X protein. Using PBMCs as antigen presenting cells, the NLGN4X¹³¹⁻¹³⁹-induced luminescence signal intensity was similar to TCR stimulation by anti-CD3/CD28 beads (FIG. 3D).

### 2.4 Generation and Phenotypic Characterization of NLGN4X¹³¹⁻¹³⁹-Reactive TCR-Transduced Human T Cells

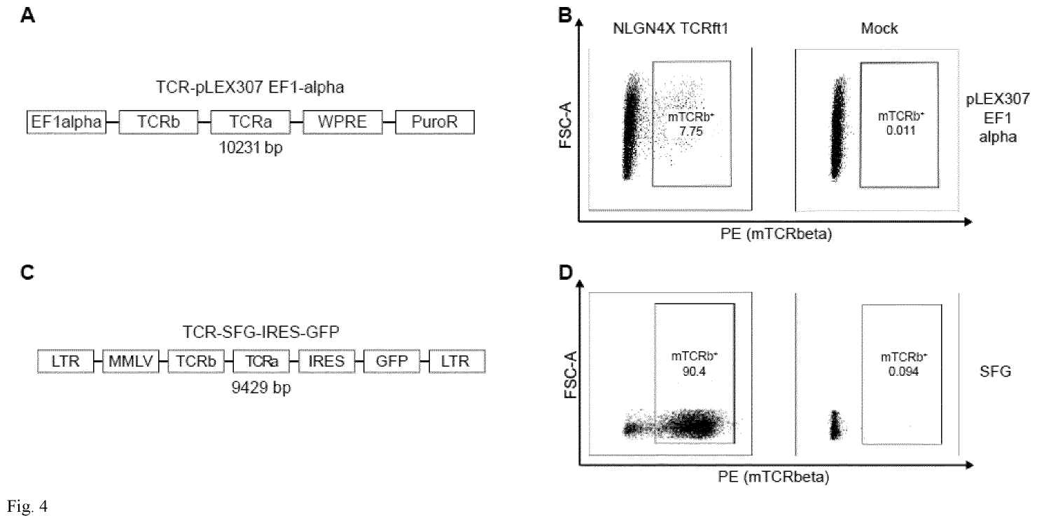

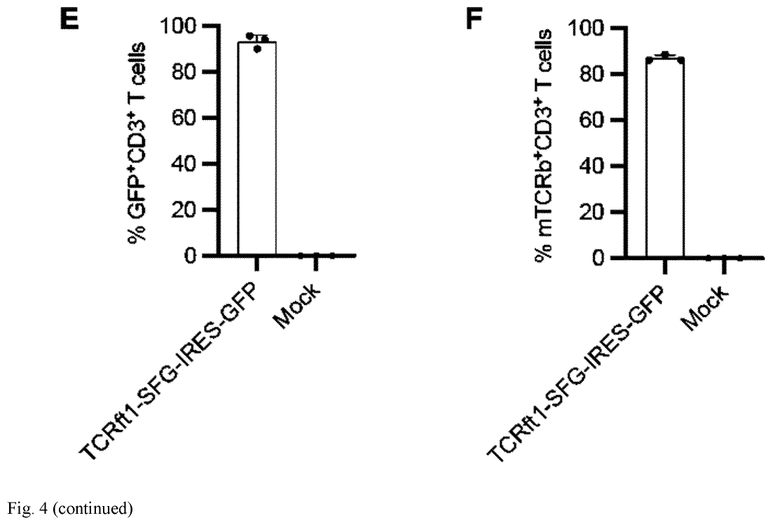

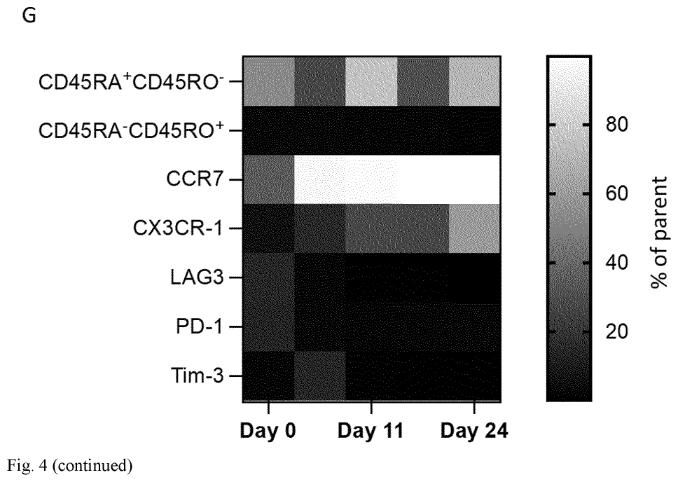

Ultimately, To deliver the NLGN4X-reactive TCR, a retroviral SFG-IRES-GFP vector (FIG. 4C) was selected, that is currently investigated in a clinical trial evaluating anti-CD19 CAR T cells in patients with relapsed or refractory CD19⁺ lymphoid disease, and was used with RD-114 and PeqPam a different packaging plasmid system. Importantly, the transduction process of primary human T cells using the retroviral TCRft1-SFG-IRES-GFP vector (FIG. 4C) resulted in up to 93.2% of GFP⁺ CD3⁺ T cells and led to a transduction efficiency of up to 90.4% (FIG. 4D-F). Consequently, the SFG-IRES-GFP vector was used for all subsequent in vitro and in vivo experiments. Prior to re-infusion, engineered autologous T cells are routinely expanded after transduction. To assess the phenotype of TCRft1-SFG-IRES-GFP-expressing primary human T cells during in vitro expansion, the TCR-transduced T cells were subjected to longitudinal flow cytometric analysis by using phenotypic markers for T cell differentiation. TCRft1-SFG-IRES-GFP-expressing T cells acquired a CD45RA⁺ CD45RO⁻ CCR7⁺ T cell phenotype (FIG. 4G) that resembles naïve T cell states and suggests that transduction with TCRft1-SFG-IRES-GFP does not lead to differentiation of T cells into terminal effector cells. Moreover, a relevant increase in expression of the exhaustion markers PD-1 and TIM-3 was not observed. Collectively, the flow cytometric profiling suggests that transduced TCRft1-SFG-IRES-GFP primary human T cells display a naïve T cell phenotype that has recently been described to provide superior anti-tumor capacities in Vitanza NA et al. (2021).

### 2.5 NLGN4X-TCR-T Lyse Glioma Cells In Vitro

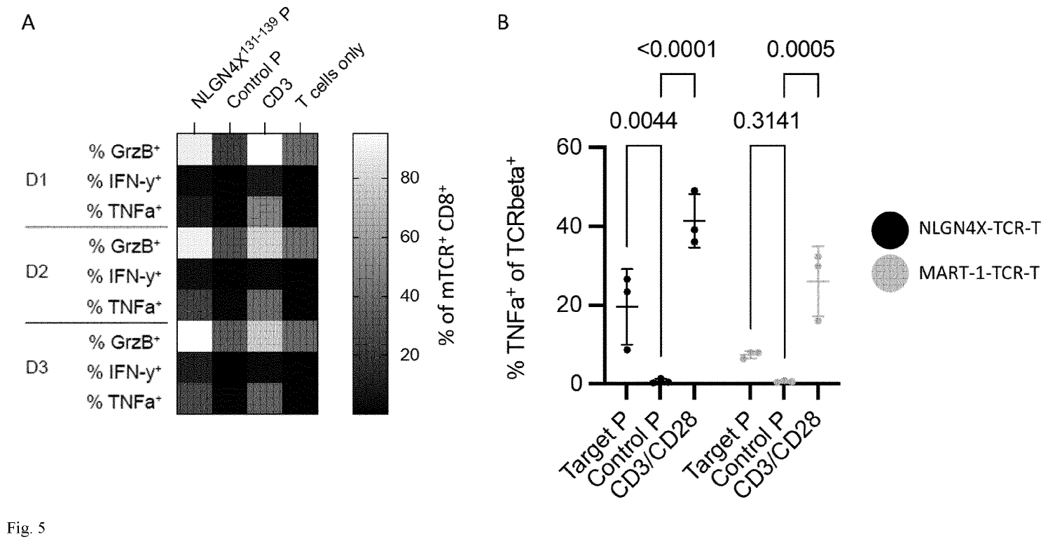

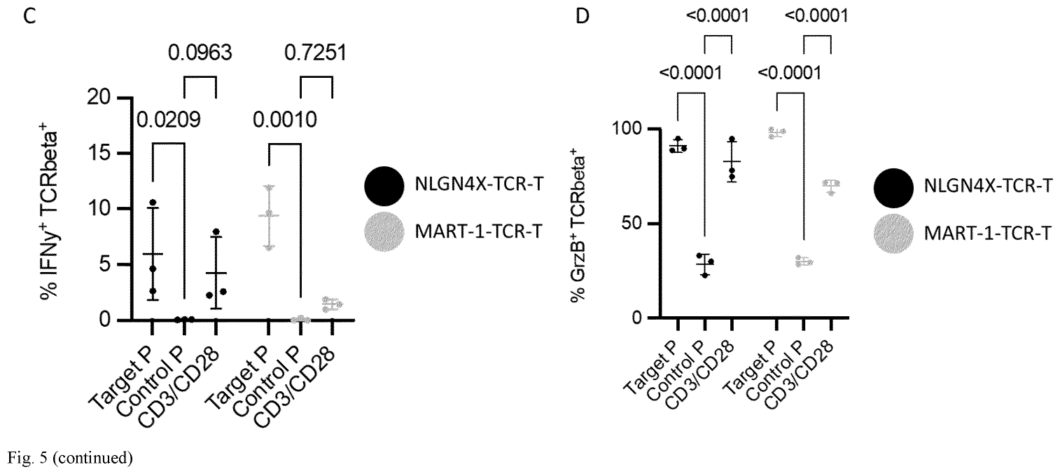

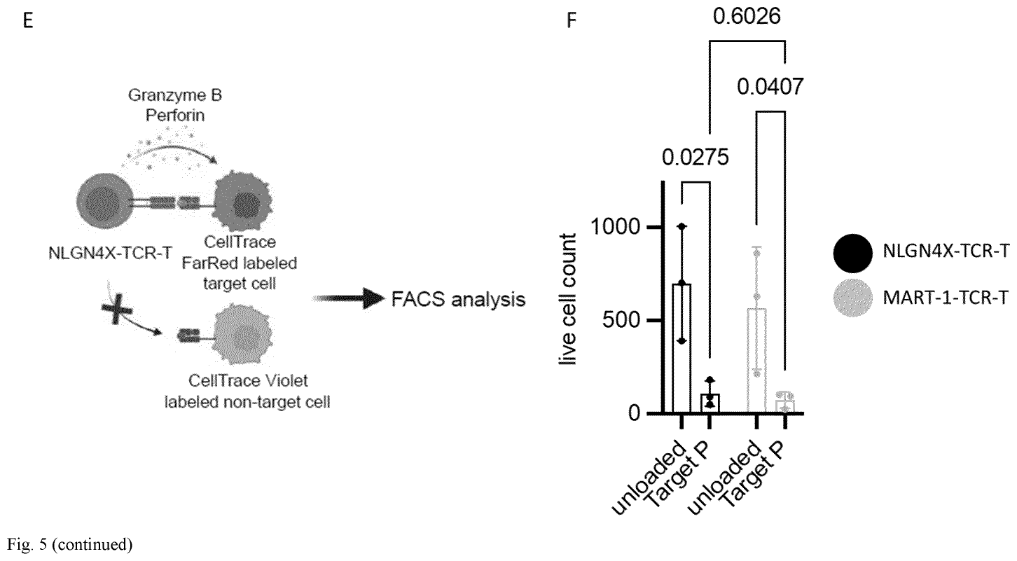

It was then aimed to assess functionality of NLGN4X¹³¹⁻¹³⁹-specific TCR-expressing primary human T cells in vitro using flow cytometric profiling following co-culture with exogenously peptide-loaded non-adherent HLA-A*02⁺ K562 leukemia cells. As most relevant effector proteins, Granzyme-B (GrzB), interferon-γ (IFN-β), and tumor necrosis factor-α (TNF-α) production was assessed. Independent of healthy T cell donors, robust expression of the highly cytotoxic cytokines TNF-α and GrzB by NLGN4X¹³¹⁻¹³⁹-specific TCR-expressing T cells (FIG. 5A-D) was found. Strikingly, when benchmarking NLGN4X¹³¹⁻¹³⁹-specific TCR-expressing primary human T cells with the well-established high-affinity melanoma antigen recognized by T cells 1 (MART-1)-specific TCR that has been used for phase I and II clinical trials, similar expression levels of GrzB, IFN-γ, and TNF-α in the co-culture systems (FIG. 5B-D) were observed. In order to confirm that the robust expression of cytotoxic proteins by NLGN4X¹³¹⁻¹³⁹-specific TCR-expressing primary human T cells leads to target cell killing, a modified version of the Vital-FR assay was utilized to detect specific lysis of peptide-loaded K562 target cells (FIG. 5E). Importantly, NLGN4X¹³¹⁻¹³⁹-specific and MART1-specific T cells (MART-1-TCR-T) showed comparable cytotoxic activity against peptide-loaded K562 target cells (FIG. 3F).

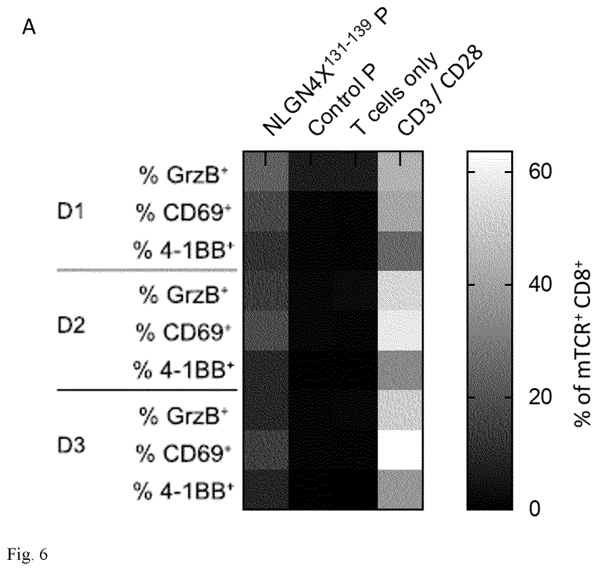

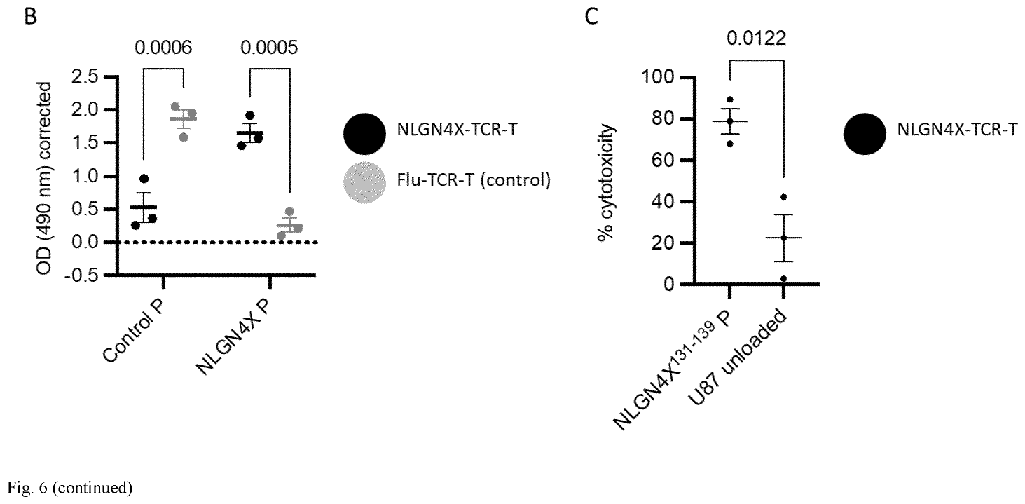

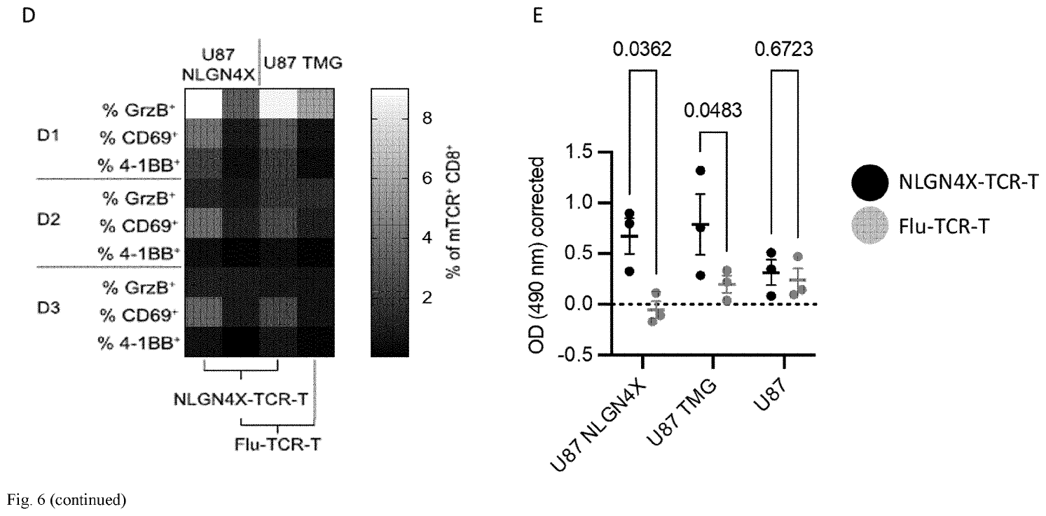

Next, it was aimed to evaluate the cytotoxic capacity of NLGN4X-TCR-T against the HLA-A*02⁺ adherent glioma cell line U87. U87 wildtype cells loaded with the NLGN4X¹³¹⁻¹³⁹ peptide induced specific upregulation of GrzB, CD69, and 4-1BB (CD137) in NLGN4X-TCR-T (FIG. 6A). In line with these findings, NLGN4X¹³¹⁻¹³⁹-specific T cells were able to specifically lyse U87 cells loaded with the target peptide in lactate dehydrogenase (LDH)-based and flow cytometry-based killing assays (FIG. 6B, C). In co-culture assays applying synthetic MHC class I-restricted peptides for reactivity testing, MHC molecules are usually saturated and external MHC loading might occur. Although the NLGN4X¹³¹⁻¹³⁹. was previously identified by HLA-ligandome analysis in some glioblastoma patients¹, it was aimed at demonstrating endogenous presentation of NLGN4X¹³¹⁻¹³⁹ in the tumor model system. Thus, the NLGN4X¹³¹⁻¹³⁹ antigen was expressed by using either a tandem-minigene (TMG) with different MHC class I epitopes including NLGN4X (U87 TMG) or a retroviral vector containing full-length NLGN4X (U87 NLGN4X). Similar to the findings with K562 cells as target cells, specific upregulation of GrzB, CD69, and 4-1BB was found when NLGN4X-TCR-T was co-cultured with U87 NLGN4X or U87 TMG target cells (FIG. 6D). Moreover, U87 TMG and U87 NLGN4X were specifically lysed by NLGN4X-TCR-T (FIG. 6E). In summary, it was demonstrated that TCRft1-SFG-IRES-GFP-expressing primary human T cells specifically recognize and lyse tumor cells expressing the NLGN4X¹³¹⁻¹³⁹ epitope in vitro.

### 2.6 NLGN4X-TCR-T Promote Tumor Regression and Improve Survival of HLA-A*02⁺ Glioma-Bearing Mice

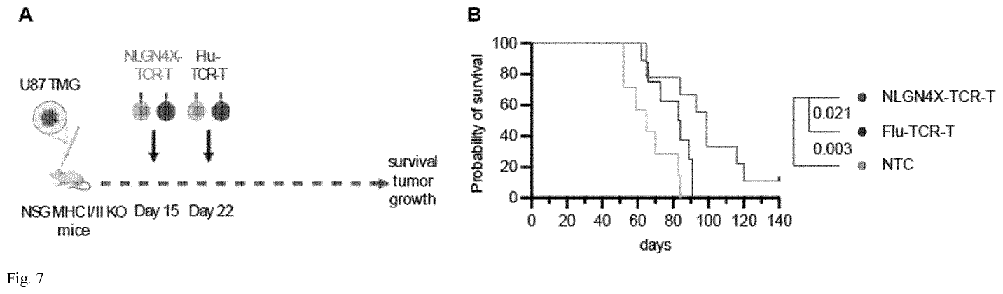

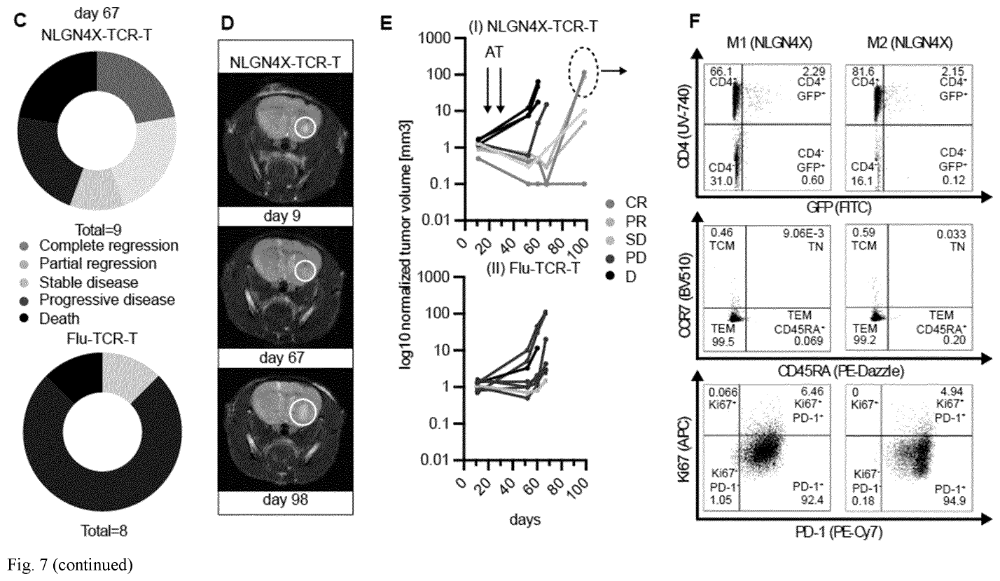

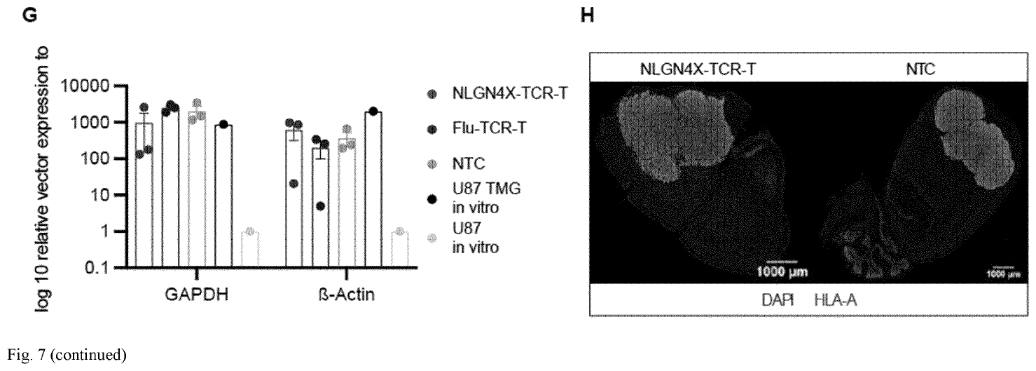

Having demonstrated specific recognition of the NLGN4X¹³¹⁻¹³⁹ epitope and tumor cell lysis of the NLGN4X-expressing U87 human glioma cell line by NLGN4X¹³¹⁻¹³⁹-specific TCR-engineered human T cells, next it was aimed to assess their therapeutic potential in vivo. Therefore, NOD scid gamma (NSG) MHC class I and MHC class II knockout (NSG MHCI/II KO) mice, that do not develop graft versus host disease after T cell transfer, were challenged with intracranial U87 TMG experimental gliomas. Mice received either NLGN4X¹³¹⁻¹³⁹-specific TCR- or negative control ((influenza (Flu)) TCR-engineered human T cells (Flu-TCR-T) via intracerebroventricular transfer or did not receive any T cell treatment ((NTC)=No T cell Control) (FIG. 7A). Two injections of 5×10⁶ TCR-engineered primary human HLA-A*02⁺ T cells with 86.6 to 87.4% mTCRb surface expression after transduction were performed at day 15 and 22 (FIG. 7A) into the lateral ventricle of the non-tumor-bearing hemisphere. Treatment with NLGN4X-TCR-T resulted in prolonged survival of glioma-bearing animals compared to Flu-TCR-T or NTC mice (FIG. 7B). By using longitudinal MRI, it was aimed to investigate if treatment with NLGN4X-TCR-T leads to objective radiographic responses, therefore tumor volumes between day 11 and day 67 according to the modified RANO criteria were assessed. At day 67 as the timepoint of best response, stable disease in 11.1%, partial responses (PR) in 22.2% and complete response (CR) in 22.2% of NLGN4X-TCR-T treated mice resulting in an objective response rate (ORR: CR+PR) of 44.4% in comparison to 0.0% in both Flu-TCR-T and NTC mice (FIG. 7C) were observed. In addition to the assessment of radiographic responses upon NLGN4X¹³¹⁻¹³⁹-specific TCR-engineered T cell therapy, longitudinal MRI enabled the local assessment of tumor growth (FIG. 5D, E). Interestingly, two mice with late recurrence of U87 TMG tumors (FIG. 7E) had shown radiographic responses (one PR, one CR) in previous MRI (FIG. 7E). Thus, flow cytometric analysis of tumor-infiltrating leukocytes from recurrent late-stage tumors (FIG. 7F) was performed. In these tumors, 78 days after the second administration of NLGN4X-TCR-T, predominantly CD4⁺ T cells with low GFP expression within the experimental tumors were found. These CD4⁺ T cells were CD45RA CCR7 and had low expression of the proliferation marker Ki67 and high PD-1 expression (FIG. 7F).

Notably, recurrent U87 TMG tumors maintained expression of the NLGN4X¹³¹⁻¹³⁹ antigen as demonstrated by qPCR and in addition, MHC class I expression was still detectable by immunofluorescent staining (FIG. 7G, H). Overall, these findings suggest that late recurrence results from the absence of intratumoral cytotoxic NLGN4X¹³¹⁻¹³⁹-specfic CD8⁺ T cells (FIG. 7F-H).

### 2.7 NLGN4X-TCR-T Phenotypically Adapt Within the Tumor Microenvironment

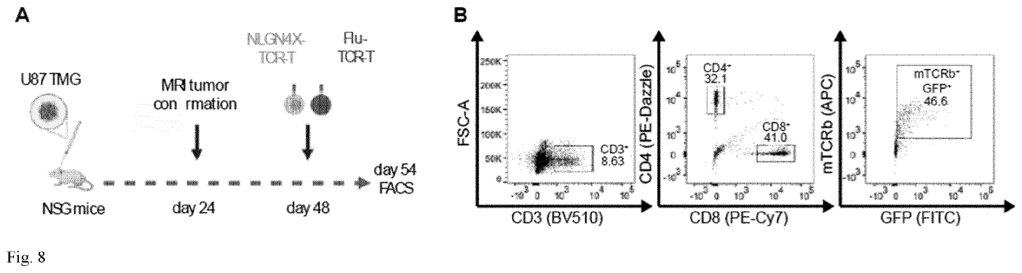

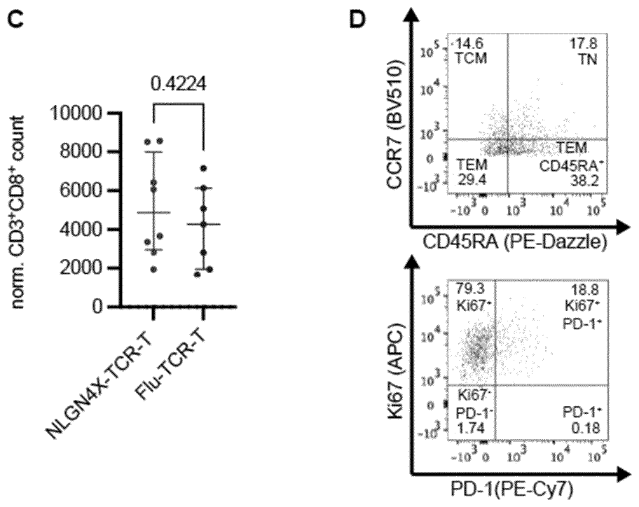

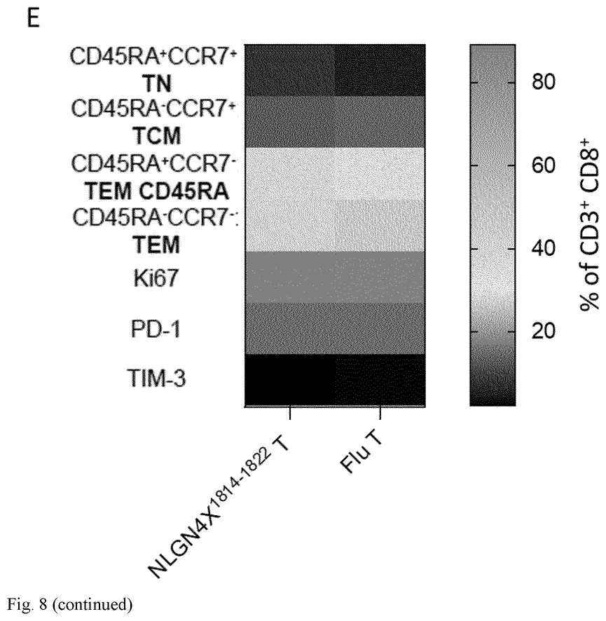

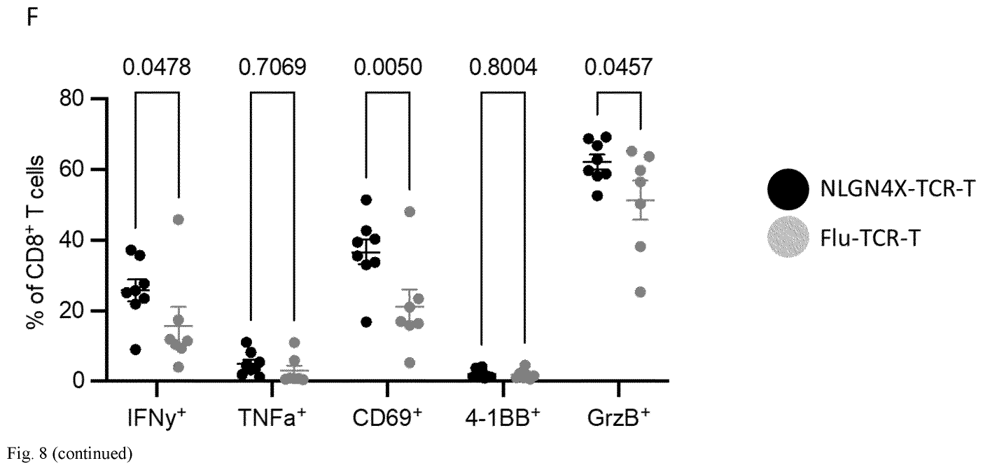

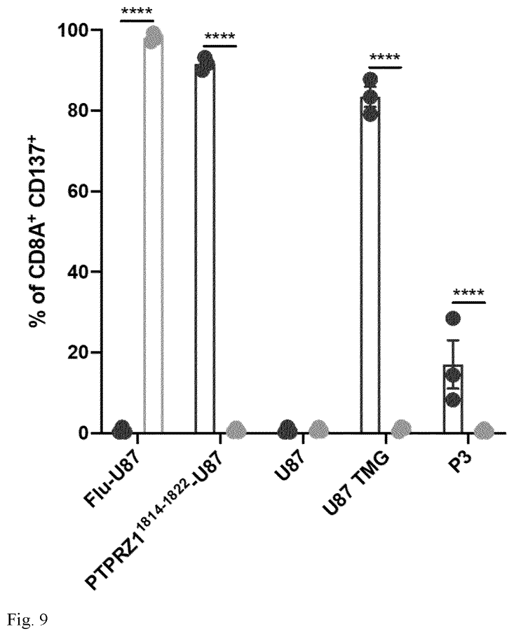

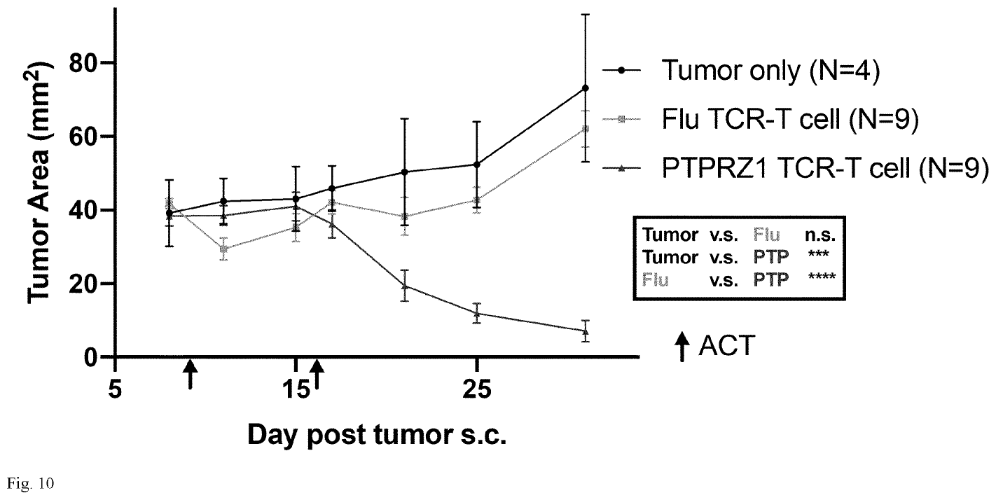

At late recurrence, exhausted CD4⁺ T cells with low TCR transgene expression were found. Hence, this observation prompted assessment of the intratumoral phenotype of NLGN4X-TCR-T at an early timepoint after intracerebroventricular delivery in U87 TMG glioma-bearing animals (FIG. 8A). 6 days after intracerebroventricular transfer, T cells were present in the contralateral experimental glioma and NLGN4X¹³¹⁻¹³⁹-specific TCR positivity was confirmed on the CD8⁺ T cell subset (FIG. 8B). Quantitatively, U87 TMG gliomas treated with NLGN4X-TCR-T and Flu-TCR-T were not differentially infiltrated by CD3⁺ T cells in immunocompromised mice (FIG. 8C). CD8⁺ T cells mainly showed a T effector memory (TEM) phenotype with expression of CD45RA and high expression of Ki67 compared to low expression of the exhaustion markers PD-1 and TIM-3 (FIG. 8D, E). Specific upregulation of various activation and effector cell markers in NLGN4X-TCR-T compared to Flu-TCR-T (FIG. 8F), suggests antigen recognition and specific intratumoral T cell activation. However, the fundamental difference between the early phenotypes of intratumoral NLGN4X-TCR-T (FIG. 8) and those found at late recurrence (FIG. 7E) support the hypothesis that loss of cytotoxic CD8⁺ NLGN4X-TCR-T results in late tumor recurrence. Altogether, it was demonstrated that human T cells engineered with a patient-retrieved off-the-shelf TCR targeting the NLGN4X¹³¹⁻¹³⁹ antigen are capable of lysing tumor cells in vitro and mediating temporary tumor control in experimental gliomas.

### LITERATURE

Hilf et al., Nature 565:240-245 (2019);
Keskin et al., Nature 565:234-239 (2019);
Platten et al., Nature 592:463-468 (2021);
Ellingson B M et al., Neurotherapeutics, 14 (2): 307-320 (2017);
Rohaan M W et al., N engl J Med., 387 (23): 2113-2125 (2022);
Morgan R A et al., J Immunother., 36 (2): 133-51 (2013);
Chheda Z S et al., J Exp Med., 215 (1): 141-157 (2018);
Immisch L et al., J Immunother Cancer, October 2022; 10 (10);
Kilian M et al., Clin Cancer Res. 28 (2): 378-389 (2022);
Bolinger M F et al., Proc NAI Acad Sci USA, 105 (17): 6421-6 (2008);
Choe J H et al., Sci Transl Med, 28 2021; 13 (591) (2021);
Sahillioglu A C et al., Curr Opin Immunol., 74:190-198 (2022);
Choo J et al., J Virol., 88 (18): 10613-23 (2014);
Vitanza N A et al., Nat Med, 27 (9): 1544-1552 (2021).

1-15. (canceled)

16. A binding polypeptide comprising a first variable T cell receptor (TCR) domain and a second variable TCR domain, wherein:
(i) the complementarity determining region 3 (CDR3) of the first variable TCR domain comprises the amino acid sequence of SEQ ID NO:1 or a sequence at least 80% identical thereto; and/or wherein the CDR3 of the second variable TCR domain comprises the amino acid sequence of SEQ ID NO:2 or a sequence at least 80% identical thereto; or
(ii) the CDR3 of the first variable TCR domain comprises the amino acid sequence of SEQ ID NO:3 or a sequence at least 80% identical thereto; and/or wherein the CDR3 of the second variable TCR domain comprises the amino acid sequence of SEQ ID NO:4 or a sequence at least 80% identical thereto.

17. The binding polypeptide of claim 16, wherein:
(i) the first variable TCR domain comprises the amino acid sequence of SEQ ID NO: 5 or an amino acid sequence at least 70% identical to SEQ ID NO:5 and/or wherein the second variable TCR domain comprises the amino acid sequence of SEQ ID NO:6 or an amino acid sequence at least 70% identical to SEQ ID NO:6; or
(ii) the first variable TCR domain comprises the amino acid sequence of SEQ ID NO: 7 or an amino acid sequence at least 70% identical to SEQ ID NO:7 and/or wherein the second variable TCR domain comprises the amino acid sequence of SEQ ID NO:8 or an amino acid sequence at least 70% identical to SEQ ID NO:8.

18. The binding polypeptide of claim 16, wherein:
the binding polypeptide recognizes a peptide having the amino acid sequence MIWEHNVEV (SEQ ID NO:14) when presented on an MHC class I molecule; or
the binding polypeptide recognizes a peptide having the amino acid sequence NLDTLMTYV (SEQ ID NO:13) when presented on an MHC class I molecule.

19. The binding polypeptide of claim 18, wherein the MHC class I molecule is an HLA-A molecule.

20. The binding polypeptide of claim 18, wherein the MHC class I molecule is an HLA-A*02 molecule.

21. The binding polypeptide of claim 16, wherein the binding polypeptide is a TCR.

22. The binding polypeptide of claim 16, wherein the binding polypeptide is a chimeric antigen receptor (CAR).

23. The binding polypeptide of claim 16, wherein the first variable TCR domain and the second variable TCR domain are covalently connected to an Fc domain of an immunoglobulin; or wherein said binding polypeptide is a soluble TCR.

24. A polynucleotide encoding at least one of a first variable TCR domain and a second variable TCR domain of a binding polypeptide according to claim 16.

25. A method of treating and/or preventing cancer in a subject, the method comprising:
(a) contacting the subject with a binding polypeptide according to claim 16, and
(b) thereby treating and/or preventing cancer in the subject.

26. A method of identifying a cancer susceptible to treatment with a binding polypeptide according to claim 16, comprising:
(i) identifying target cells expressing PTPRZ1 and/or NLGN4X in a cancer sample; and
(ii) identifying a cancer susceptible to treatment based on the identifying in step (i).

27. The method of claim 26, wherein identifying target cells expressing PTPRZ1 and/or NLGN4X comprises:
(I) contacting the target cells with a binding polypeptide comprising a first variable T cell receptor (TCR) domain and a second variable TCR domain, wherein:
(a) the complementarity determining region 3 (CDR3) of the first variable TCR domain comprises the amino acid sequence of SEQ ID NO:1 or a sequence at least 80% identical thereto; and/or wherein the CDR3 of the second variable TCR domain comprises the amino acid sequence of SEQ ID NO:2 or a sequence at least 80% identical thereto; or
(b) the CDR3 of the first variable TCR domain comprises the amino acid sequence of SEQ ID NO:3 or a sequence at least 80% identical thereto; and/or wherein the CDR3 of the second variable TCR domain comprises the amino acid sequence of SEQ ID NO:4 or a sequence at least 80% identical thereto;
(II) detecting binding of the binding polypeptide to the target cell; and
(III) based on the detecting in step (II), identifying a target cell expressing PTPRZ1 and/or NLGN4X.

28. The method of claim 25, wherein the cancer is a brain cancer.

29. The method of claim 25, wherein the cancer is a glioma.

30. The method of claim 25, wherein the cancer is a glioblastoma.

31. The method of claim 26, wherein the cancer is a brain cancer.

32. The method of claim 26, wherein the cancer is a glioma.

33. The method of claim 26, wherein the cancer is a glioblastoma.


SEQUENCE LISTING (14 sequences)

SEQ ID NO: 1 - protein, 12 aa, synthetic construct
  REGION 1..12: note = CDR3a PTPRZ 1(1814-1822)
AVSDRGAGNMLT

SEQ ID NO: 2 - protein, 15 aa, synthetic construct
  REGION 1..15: note = CDR3b PTPRZ1(1814-1822)
ASSLGLAGGLAGELF

SEQ ID NO: 3 - protein, 12 aa, synthetic construct
  REGION 1..12: note = CDR3a NLGN4X(131-139)
VVTDSGNTGKLI

SEQ ID NO: 4 - protein, 10 aa, synthetic construct
  REGION 1..10: note = CDR3b NLGN4X(131-139)
SARVDYNEQF

SEQ ID NO: 5 - protein, 133 aa, synthetic construct
  REGION 1..133: note = VDJ alpha PTPRZ1(1814-1822)
MLLLLVPAFQVIFTLGGTRAQSVTQLDSQVPVFEEAPVELRCNYSSSVSVYLFWYVQYPNQGLQLLLKYLSGSTLVKGINGFEAEFNKSQTSFHLRKPSVHISDTAEYFCAVSDRGAGNMLTFGGGTRLMVKP

SEQ ID NO: 6 - protein, 136 aa, synthetic construct
  REGION 1..136: note = VDJ beta PTPRZ1(1814-1822)
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLGLAGGLAGELFFGEGSRLTVL

SEQ ID NO: 7 - protein, 132 aa, synthetic construct
  REGION 1..132: note = VDJ alpha NLGN4X(131-139)
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVTDSGNTGKLIFGQGTTLQVKP

SEQ ID NO: 8 - protein, 147 aa, synthetic construct
  REGION 1..147: note = VDJ beta NLGN4X(131-139)
MEAVVTTLPREGGVRPSRKMLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARVDYNEQFFGPGTRLTVL

SEQ ID NO: 9 - DNA, 400 bp, synthetic construct
  misc_feature 1..400: note = PTPRZ1(1814-1822) VDJ incl CDR3a encoding
  source 1..400: mol_type = other DNA
atgctcctgctgctcgtcccagcgttccaggtgatttttaccctgggaggaaccagagcccagtctgtgacccagcttgacagccaagtccctgtctttgaagaagcccctgtggagctgaggtgcaactactcatcgtctgtttcagtgtatctcttctggtatgtgcaataccccaaccaaggactccagcttctcctgaagtatttatcaggatccaccctggttaaaggcatcaacggttttgaggctgaatttaacaagagtcaaacttccttccacttgaggaaaccctcagtccatataagcgacacggctgagtacttctgtgctgtgagtgatcgaggtgcaggcaacatgctcacctttggagggggaacaaggttaatggtcaaacccc

SEQ ID NO: 10 - DNA, 409 bp, synthetic construct
  misc_feature 1..409: note = PTPRZ1(1814-1822) VDJ incl CDR3b encoding
  source 1..409: mol_type = other DNA
atgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtctccactctgacgatccagcgcacacagcaggaggactcggccgtgtatctctgtgccagcagcttaggactagcggggggactcgccggggagctgttttttggagaaggctctaggctgaccgtactgg

SEQ ID NO: 11 - DNA, 397 bp, synthetic construct
  misc_feature 1..397: note = NLGN4X(131-139) VDJ incl CDR3a encoding
  source 1..397: mol_type = other DNA
atgatatccttgagagttttactggtgatcctgtggcttcagttaagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgaccgactcgggcaacacaggcaaactaatctttgggcaagggacaactttacaagtaaaaccag

SEQ ID NO: 12 - DNA, 442 bp, synthetic construct
  misc_feature 1..442: note = NLGN4X(131-139) VDJ incl CDR3b encoding
  source 1..442: mol_type = other DNA
atggaggcagtggtcacaactctccccagagaaggtggtgtgaggccatcacggaagatgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagtggattacaatgagcagttcttcgggccagggacacggctcaccgtgctag

SEQ ID NO: 13 - protein, 9 aa, synthetic construct
  REGION 1..9: note = NLGN4X(131-139) peptide
NLDTLMTYV

SEQ ID NO: 14 - protein, 9 aa, synthetic construct
  REGION 1..9: note = PTPRZ1(1814-1822) peptide
MIWEHNVEV